SEQUENCE LISTING (12 sequences)

SEQ ID NO: 1 - protein, 5 aa, synthetic construct
  REGION 1..5: note = HCDR1 of Daratumumab
SFAMS

SEQ ID NO: 2 - protein, 17 aa, synthetic construct
  REGION 1..17: note = HCDR2 of Daratumumab
AISGSGGGTYYADSVKG

SEQ ID NO: 3 - protein, 13 aa, synthetic construct
  REGION 1..13: note = HCDR3 of Daratumumab
DKILWFGEPVFDY

SEQ ID NO: 4 - protein, 11 aa, synthetic construct
  REGION 1..11: note = LCDR1 of Daratumumab
RASQSVSSYLA

SEQ ID NO: 5 - protein, 7 aa, synthetic construct
  REGION 1..7: note = LCDR2 of Daratumumab
DASNRAT

SEQ ID NO: 6 - protein, 10 aa, synthetic construct
  REGION 1..10: note = LCDR3 of Daratumumab
QQRSNWPPTF

SEQ ID NO: 7 - protein, 122 aa, synthetic construct [sequence omitted]

SEQ ID NO: 8 - protein, 107 aa, synthetic construct [sequence omitted]

SEQ ID NO: 9 - protein, 452 aa, synthetic construct
  REGION 1..452: note = HC of Daratumumab
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  REGION 1..214: note = LC of Daratumumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

(19) United States
(12) Patent Application Publication
(10) Pub. No.: US 2023/0348615 A1
(43) Pub. Date: Nov. 2, 2023

(54) Clinically Proven Subcutaneous Pharmaceutical Compositions Comprising Anti-CD38 Antibodies and Their Uses

(71) Applicant: Janssen Biotech, Inc., Horsham, PA (US)

(72) Inventors: Rajesh Bandekar, Collegeville, PA (US); Pamela Clemens, Verona, WI (US); Christoph Heuck, Princeton, NJ (US); Ming Qi, Phoenixville, PA (US); Zhilong Yuan, Hillsborough, NJ (US)

(21) Appl. No.: 18/348,384

(22) Filed: Jul. 7, 2023

Related U.S. Application Data
(63) Continuation of application No. 17/475,975, filed on Sep. 15, 2021.
Application No. 17/475,975 is a continuation of application No. 16/830,629, filed on Mar. 26, 2020, now abandoned.
(60) Provisional application No. 62/825,244, filed on Mar. 28, 2019.

(51) Int. Cl. C07K 16/28; A61K 38/47; A61K 47/18; A61K 47/22; A61K 47/26; A61P 35/00; C12N 9/24; A61K 47/10

(52) U.S. Cl. CPC C07K 16/2896; A61K 38/47; A61K 47/183; A61K 47/22; A61K 47/26; A61P 35/00; C12N 9/2402; A61K 47/10; A61K 45/06

(57) ABSTRACT

The present invention relates to clinically proven subcutaneous pharmaceutical compositions comprising anti-CD38 antibodies and their uses.

### CROSS-REFERENCE TO RELATED APPLICATIONS

This application is a Continuation of U.S. application Ser. No. 17/475,975, filed Sep. 15, 2021, which is a Continuation of U.S. application Ser. No. 16/830,629, filed Mar. 26, 2020, now abandoned, which claims the benefit of U.S. Provisional Application Ser. No. 62/825,244, filed Mar. 28, 2019, the entire contents of which are incorporated herein by references in their entireties.

### FIELD OF THE INVENTION

The present invention relates to clinically proven subcutaneous pharmaceutical compositions comprising anti-CD38 antibodies and their uses.

### SEQUENCE LISTING

The instant application contains a Sequence Listing which is being submitted herewith electronically in XML format and is hereby incorporated by reference in its entirety. Said XML copy, created on Jul. 6, 2023, is named JBI6060USCNT2_SL.xml and is 17,024 bytes in size.

### BACKGROUND OF THE INVENTION

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J Immunol 145:2390-6, 1990; Terhorst et al., Cell 771-80, 1981; Guse et al., Nature 398:70-3, 1999). CD38, via its NAD glycohydrolase activity, also regulates extracellular NAD⁺ levels, which have been implicated in modulating the regulatory T-cell compartment (Adriouch et al., 14:1284-92, 2012; Chiarugi et al., Nature Reviews 12:741-52, 2012). In addition to signaling via Ca²⁺, CD38 signaling occurs via cross-talk with antigen-receptor complexes on T- and B-cells or other types of receptor complexes, e.g., MHC molecules, involving CD38 in several cellular responses, but also in switching and secretion of IgG1. CD38 is expressed on various malignant cells.

Anti-CD38 antibodies are being developed for the treatment of multiple myeloma and other heme malignancies. The antibodies infused via the intravenous (IV) route. The amount of antibody that can be administered via the IV route is limited by the physico-chemical properties of the antibody, in particularly by its solubility and stability in a suitable liquid formulation and by the volume of the infusion fluid.

Therefore, there is a need for additional anti-CD38 antibody formulations and pharmaceutical compositions.

### SUMMARY OF THE INVENTION

The disclosure provides a method of treating a subject having a disease involving cells expressing CD38 who is expected to benefit from a treatment with an antibody that specifically binds CD38, comprising:
providing a healthcare professional a pharmaceutical composition comprising an antibody that specifically binds CD38 comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and recombinant human hyaluronidase (rHuPH20), wherein the pharmaceutical composition is clinically proven for subcutaneous administration;
providing the healthcare professional information that the pharmaceutical composition is clinically proven for subcutaneous administration; wherein performing the steps a) and b) results in the medical professional to administer subcutaneously the pharmaceutical composition to the subject having the disease involving cells expressing CD38, thereby treating the subject having the disease involving cells expressing CD38.

The disclosure also provides a method of reducing occurrence or severity of infusion related reactions (IRR) in a subject who is treated with an antibody that specifically binds CD38, comprising
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration;
providing the healthcare professional information that the pharmaceutical composition is clinically proven for subcutaneous administration and that subcutaneous administration of the pharmaceutical composition has been demonstrated to result in reduced occurrence or severity of IRR when compared to intravenous administration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6; wherein performing the steps a) and b) results in the healthcare professional to administer subcutaneously the pharmaceutical composition to the subject, thereby reducing occurrence or severity of IRR in the subject.

The disclosure also provides a method of treating a subject having a disease involving cells expressing CD38 who is expected to benefit from a treatment with an antibody that specifically binds CD38, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration.

The disclosure also provides a method of reducing occurrence or severity of infusion related reactions (IRR) in a subject who is treated with an antibody that specifically binds CD38, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration.

The disclosure also provides a method of treating a subject with multiple myeloma, comprising:
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration; providing the healthcare professional information that the pharmaceutical composition is clinically proven for subcutaneous administration; wherein performing the steps a) and b) results in the medical professional to administer subcutaneously the pharmaceutical composition to the subject having multiple myeloma, thereby treating the subject having multiple myeloma.

The disclosure also provides a method of treating a subject with multiple myeloma, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration.

The disclosure also provides a pharmaceutical composition comprising a clinically proven amount of an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is intended for subcutaneous administration.

The disclosure also provides a pharmaceutical composition for subcutaneous administration comprising a clinically proven amount of an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20.

### BRIEF DESCRIPTION OF THE DRAWINGS

FIG. 1 shows the summary of overall best response in DARZALEX® (daratumumab) intravenous (DARA IV) and daratumumab subcutaneous (DARA SC) groups, intent-to-treat population. ORR: overall response rate; ≥VGPR: very good partial response or better rate; ≥CR: complete response or better rate.

FIG. 2 shows the maximum Ctrough concentration (μg/mL) in DARZALEX® (daratumumab) intravenous (DARA IV) and daratumumab subcutaneous (DARA SC) groups, PK-evaluable population.

FIG. 3 shows the Kaplan-Meier plot for progression-free survival (PFS) in DARZALEX® (daratumumab) intravenous (DARA IV) and daratumumab subcutaneous (DARA SC) groups, intent-to-treat population.

FIG. 4 shows the Kaplan-Meier plot for overall survival (OS) in DARZALEX® (daratumumab) intravenous (DARA IV) and daratumumab subcutaneous (DARA SC) groups, intent-to-treat population.

FIG. 5 shows the incidence rate of treatment-emergent adverse events (TEAE) in DARZALEX® (daratumumab) intravenous (DARA IV) and daratumumab subcutaneous (DARA SC) groups, safety analysis population. AE: adverse event; SAE: serious adverse event; IRR: infusion related reaction; IJSR: injection site related reaction.

FIG. 6 shows the most commonly reported treatment-emergent adverse events in DARZALEX® (daratumumab) intravenous (DARA IV) and daratumumab subcutaneous (DARA SC) groups, safety analysis population.

### DETAILED DESCRIPTION OF THE INVENTION

### Definitions

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“About once a week” refers to an approximate number, and can include every 7 days±two days, i.e., every 5 days to every 9 days. The dosing frequency of “once a week” thus can be every five days, every six days, every seven days, every eight days, or every nine days.

“About once in two weeks” refers to an approximate number, and can include every 14 days±two days, i.e., every 12 days to every 16 days.

“About once in three weeks” refers to an approximate number, and can include every 21 days±two days, i.e., every 19 to every 23 days.

“About once in four weeks” refers to an approximate number, and can include every 28 days±two days, i.e., every 26 to every 30 days.

“About once in five weeks” refers to an approximate number, and can include every 35 days±two days, i.e., every 33 to every 37 days.

“About once in six weeks” refers to an approximate number, and can include every 42 days±two days, i.e., every 40 to every 38 days.

“About twice a week” refers to an approximate number, can include twice in one week, e.g., a first dose on day 1 and a second dose on day 2, day 3, day 4, day 5, day 6 or day 7 of the week, the first dose on day 2 and the second dose on day 3, day 4, day 5, day 6 or day 7 of the week, the first dose on day 3 and the second dose on day 4, day 5, day 6 or day 7 of the week, the first dose on day 4 and the second dose on day 5, day 6 or day 7 of the week, the first dose on day 5 and the second dose on day 6 or day 7 of the week, the first dose on day 6 and the second dose on day 7 of the week.

“Adverse event” or “AE” refers to any untoward medical occurrence in a clinical study subject administered an antibody that specifically binds CD38. An AE does not necessarily have a causal relationship with the treatment. An AE can therefore be any unfavorable and unintended sign (including an abnormal finding), symptom, or disease temporally associated with the use of a medicinal (investigational or non-investigational) product, whether or not related to the antibody that specifically binds CD38.

“Alkylating agent” refers to family of DNA alkylating agents including cyclophosphamide, ifosfamide, melphalan or nitrosoureas. Cyclophosphamide is marketed under the trade name Cyclostin™. Ifosfamide is marketed under the trade name Holoxan™. Melphalan is marketed under the trade name ALKERAN®. Nitrosureas include carmustine, lomustine and semustine. Carmustine is marketed under the trade name BiCNU®. Lomustine is marketed under the trade name GLEOSTINE®.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

“Antibody” includes immunoglobulin molecules belonging to any class, IgA, IgD, IgE, IgG and IgM, or sub-class IgA1, IgA2, IgG1, IgG2, IgG3 and IgG4 and including either kappa (κ) and lambda (λ) light chain. Antibodies include monoclonal antibodies including human, humanized and chimeric monoclonal antibodies. Full-length antibody molecules are comprised of two heavy chains (HC) and two light chains (LC) inter-connected by disulfide bonds. Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDRs), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA®. Gemcitabine is marketed under the trade name GEMZAR®

“Autoimmune disease” refers to any group of disorders in which tissue injury is associated with humoral or cell-mediated responses to the body's own constituents. Thus, the term autoimmune disease encompasses disorders that result from an autoimmune response.

“Benefit from a treatment” refers to an improvement of at least one symptom or well-being of the patient and includes reduction in a tumor burden, arrested or slowed growth of a tumor, and/or absence of metastasis of cancer cells to other locations in the body.

“Biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extend the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Cancer” refers to an abnormal growth of cells which tend to proliferate in an uncontrolled way and, in some cases, to metastasize (spread) to other areas of a patient's body.

“CD38” refers to human cluster of differentiation 38 protein, a glycoprotein expressed on immune cells, including plasma cells, natural killer cells and sub-populations of B and T cells.

“Clinical efficacy endpoint” or “clinical endpoint” refers to an outcome that represents a clinical benefit, such as progression-free survival (PFS), time to disease progression (TTP), time to next treatment, overall response rate (ORR), proportion of subjects achieving partial response (PR), proportion of subjects achieving very good partial response (VGPR), proportion of subjects achieving complete response (CR), proportion of subjects achieving stringent complete response (sCR), proportion of subjects achieving a negative status for minimal residual disease (MRD), or proportion of subjects achieving both sCR and negative status for MRD.

“Clinically proven” refers to clinical efficacy results that are sufficient to meet approval standards of U.S. Food and Drug Administration (FDA), European Medicines Agency (EMA) or a corresponding national regulatory agency. For example, the clinical study may be an adequately sized, randomized, double-blinded controlled study used to clinically proven the effects of the drug and/or non-inferiority of the drug.

“Co-administration,” “administration with,” “administration in combination with,” “in combination with” or the like, encompass administration of two or more therapeutics or drugs to a single patient, and are intended to include treatment regimens in which the therapeutics or drugs are administered by the same or different route of administration or at the same or different time.

“Complementarity determining regions” (CDRs) are “antigen binding sites” in an antibody. CDRs may be defined based on sequence variability (Wu and Kabat, J Exp Med 132:211-250, 1970; Kabat et al., Sequences of Proteins of Immunological Interest, 5th Ed. Public Health Service, National Institutes of Health, Bethesda, Md., 1991) or based on alternative delineations (see Lefranc et al., Dev Comparat Immunol 27:55-77, 2003). The International ImMunoGeneTics (IMGT) database (http_//www_imgt_org) provides a standardized numbering and definition of antigen-binding sites.

“Comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristics” of the claimed invention. Embodiments described in terms of the phrase “comprising” (or its equivalents) also provide as embodiments those independently described in terms of “consisting of” and “consisting essentially of.”

“Consolidation”, “consolidation therapy” or “consolidation period” refers to a short duration of treatment given to a subject after the subject has been treated with high dose chemotherapy (HDC) and autologous stem cell transplant (ASCT); i.e., post-HDC and ASCT.

“Complete response rate or better” (CR response rate or better) refers to the proportion of subjects achieving CR or stringent complete response (sCR) during or after the treatment.

“Corticosteroid” refers to a class of steroid hormones that are produced in the adrenal cortex or produced synthetically refers to dexamethasone, methylprednisolone, prednisolone and prednisone. Dexamethasone is marketed under the trade name DECARON®.

“Cycle” refers to the administration schedule of one or more therapeutics or drugs and refers to the period of time when the one or more therapeutics or drugs is administered to a subject. Cycle may include days in which the drug is administered and periods of rest in which the drug is not administered. Cycle length may vary, and can be for example 2 weeks, 3 weeks, 28-days (or 4 weeks), 5 weeks or 6 weeks.

“Daratumumab” refers to an antibody that specifically binds CD38 comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5, a LCDR3 of SEQ ID NO: 6, a heavy chain variable region (VH) of SEQ ID NO: 7, a light chain variable region (VL) of SEQ ID NO: 8, a heavy chain (HC) of SEQ ID NO: 9 and a light chain (LC) of SEQ ID NO: 10. Daratumumab is marketed under the trade name DARZALEX®.

“Disease involving cells expressing CD38” refers to any disease in which CD38-expressing cells play a role or is suspected to play a role in the pathogenesis of the disease, or a disease in which it is desired to reduce the number of CD38-expressing cells in a human body. Exemplary diseases include cancers, inflammatory diseases and autoimmune diseases. Exemplary cancers include multiple myeloma, smoldering multiple myeloma, plasma monoclonal gammopathy of undetermined significance (MGUS), light chain amyloidosis, acute lymphoblastic leukemia (ALL), chronic lymphoblastic leukemia (CLL), acute myelogenous leukemia (AML), chronic myelogenous leukemia (CML), B cell lymphoma, T cell lymphoma, B cell leukemia, T cell leukemia, NK cell leukemia, non-Hodgkin's lymphoma, Hodgkin's Lymphoma, Burkitt's lymphoma, diffuse large B cell lymphoma, follicular lymphoma, hairy cell leukemia, mantle cell lymphoma, Waldenström's macroglobulinemia and solid tumors such as lung cancer, non-small cell lung cancer and small cell lung cancer. Exemplary inflammatory diseases or autoimmune diseases include rheumatoid arthritis, arthritis, allergic diseases, atherosclerosis, ankylosing spondylitis, atopic dermatitis, psoriasis, psoriatic arthritis, Crohn's disease, ulcerative colitis, inflammatory bowel disease, lupus, including systemic lupus erythematosus

“Dosage” refers to the information of the amount of the therapeutic or the drug to be taken by the subject and the frequency of the number of times the therapeutic is to be taken by the subject.

“Dose” refers to the amount or quantity of the therapeutic or the drug to be taken each time.

“Drug product” (DP) refers to a finished dosage form, for example, a tablet, capsule or solution that contains an active pharmaceutical ingredient (e.g., drug substance), generally, but not necessarily, in association with inactive ingredients.

“Drug substance” (DS) refers to any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure or function of the body.

“Duration of complete response” (duration of CR) refers to the time between the date of the initial documentation of CR to the date of the first documented evidence of relapse of CR or disease progression, whichever occurs first.

“Duration of response” refers to the time between the date of initial documentation of a response (partial response (PR) or better) to the date of the first documented evidence of progressive disease.

“Duration of stringent complete response” (duration of sCR) refers to the time between the date of the initial documentation of sCR to the date of the first documented evidence of relapse of sCR or disease progression, whichever occurs first.

“Effective” refers to a dose or dosage of a therapeutic or a drug (such as an antibody that specifically binds CD38) or a combination of therapeutics or drugs that provides a therapeutic effect for a given condition and administration regimen in a subject receiving or who has received the therapeutic or the drug or the combination of the therapeutics or drugs. “Effective” is intended to mean an amount sufficient to reduce and/or prevent a clinically significant deficit in the activity, function and response of the subject, or to cause an improvement in a clinically significant condition in the subject.

“Frontline” or “firstline” therapy refers to the first treatment of a disease, such as multiple myeloma, administered to the subject.

“Healthcare professional” refers to a medical doctor, a nurse, a nurse's assistant, or a person working under direct instructions by the medical doctor or the nurse, or any person working in a hospital or a place in which treatment can be provided to the subject.

“Hyaluronidase” refers to an enzyme that degrades hyaluronic acid (EC 3.2.1.35) and lowers the viscosity of hyaluronan in the extracellular matrix, thereby increasing tissue permeability. An exemplary hyaluronidase is recombinant human hyaluronidase PH20 (rHuPH20). rHuPH20 comprises an amino acid sequence of SEQ ID NO: 12. Enzymatic activity of hyaluronidase, including rHuPH20 can be defined by units per mL (U/mL) or by total enzyme activity in a particular formulation (U). The standard definition for one unit (U) of enzyme activity is the amount of enzyme that catalyzes the reaction of 1 nmol of substrate per minute.

“Glutamic acid derivative” refers to immunomodulatory drugs that are derivatives of glutamic acid such as lenalidomide, thalidomide and pomalidomide. Lenalinomide is marketed under the trade name REVLIMID®. Thalidomide is marketed under the trade name THALOMID®. Pomalidomide is marketed under the trade name POMALYST®

“High dose chemotherapy” (HDC) and “autologous stem cell transplant” (ASCT) refer to the treatment of subjects with newly diagnosed multiple myeloma who are considered fit (e.g. subjects are “eligible”). Subjects under the age of 65 years who have one or more comorbidities likely to have a negative impact on tolerability of HDC and ASCT or subjects over the age of 65 years are usually not considered eligible for HDC and ASCT due to their frail physical status which increases the risk of mortality and transplant-related complications (e.g. subjects are “ineligible”). An exemplary comorbidity is a renal dysfunction. Exemplary HDC regimens are melphalan at a dose of 200 mg/m² body surface area with dose reductions based on age and renal function, cyclophosphamide and melphalan, carmustine, etoposide, cytarabine, and melphalan (BEAM), high-dose idarubicin, cyclophosphamide, thiotepa, busulfan, and cyclophosphamide, busulfan and melphalan, and high-dose lenalidomide (Mahajan et al., Ther Adv Hematol 9:123-133, 2018). Cyclophosphamide is marketed under the trade name Cyclostin™. Melphalan is marketed under the trade name ALKERAN®. Carmustine is marketed under the trade name BiCNU®. Etoposide is marketed under the trade name VEPESID®. Cytarabine is marketed under the trade name CYTOSAR-U®. Idarubicin is marketed under the trade name IDAMYCIN®. Thitepa is marketed under the trade name THIOPLEX®. Lenalidomide is marketed under the trade name REVLIMID®.

“High risk multiple myeloma” refers to multiple myeloma that is characterized by one or more cytogenetic abnormalities del17p, t(4;14), t(14;20), t(14;16) or del13, or any combination thereof.

“Induction”, “induction therapy” or “induction period” refers to the first treatment given for a disease with the intention of reducing the amount of malignant plasma cell burden and improving the depth of response. Induction therapy may be provided prior to treatment with high dose chemotherapy (HDC) and autologous stem cell transplant (ASCT).

“Inflammatory disease” refers to a disease caused by, resulting from, or resulting in inflammation. Inflammatory disease may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes.

“Information” refers to reported results from clinical trials and can be provided in written or electronic form, or orally, or it can be available on internet.

“Infusion related reaction” (IRR) refers to any sign or symptom experienced by a subject during the administration of a drug or a therapeutic or any event occurring within 24-hours of administration. IRRs are typically classified as Grade 1, 2, 3 or 4.

“Maintenance therapy” refers to the treatment given for a disease after remission or best response is achieved, in order to prevent or delay relapse.

“Maximum C_{trough}” or “maximum trough concentration” or “maximum C_{trough} concentration” are used interchangeably and refer to the trough plasma concentration of the therapeutic or the drug, as measured at the end of a dosing interval at steady state. In the context of this disclosure, maximum C_{trough} refers to the serum predose concentration of daratumumab on Cycle 3 Day 1 of therapy.

“mg/m²” refers to dosing of a drug in milligrams per square meter of body surface area.

“Microtubule inhibitor” (MTI) refers to microtubule destabilizing compounds and microtubule polymerization inhibitors including to taxanes, such as paclitaxel and docetaxel, vinca alkaloids, such as vinblastine or vinblastine sulfate, vincristine or vincristine sulfate and vinorelbine. Paclitaxel is marketed under the trade name TAXOL®. Docetaxel is marketed under the trade name TAXOTERE®. Vinblastine sulfate is marketed under the trade name Vinblastin R.P™. Vincristine sulfate is marketed under the trade name Farmistin™. Vinorelbine is marketed under the trade name NAVELBINE®.

“Minimal residual disease” (MRD) refers to a small number of clonal multiple myeloma cells that remain in the patient after treatment and/or during remission.

“MRD negative” or “negative status for MRD” refers to a ratio of 1:1×10⁵ or less clonal multiple myeloma cells in a bone marrow aspirate sample obtained from the subject.

“MRD negativity rate” refers to the proportion of subjects assessed as MRD negative at any timepoint after the date of randomization.

“Multiple myeloma” refers to a malignant disorder of plasma cells characterized by uncontrolled and progressive proliferation of one or more malignant plasma cells. The abnormal proliferation of plasma (myeloma) cells causes displacement of the normal bone marrow leading to dysfunction in hematopoietic tissue and destruction of the bone marrow architecture, resulting in progressive morbidity and eventual mortality.

“Newly diagnosed” refers to a human subject who has been diagnosed with but has not yet received treatment for a disease, such as a disease involving cells that express CD38, such as multiple myeloma.

“Non-inferior” or “non-inferiority” means that, in randomized clinical trial, the experimental treatment is not unacceptably less efficacious than a reference approved control treatment. In the context of this disclosure, non-inferior means that the pharmaceutical composition comprising daratumumab and rhPH20 administered subcutaneously has an acceptable overall response rate (ORR) and maximum C_{trough} when compared to DARZALEX® (daratumumab) administered intravenously. Non-inferiority in terms of maximum C_{trough} is reached if the lower bound of the 90% confidence interval (CI) for the ratio of the geometric means of C_{trough} (subcutaneous: intravenous administration) on Cycle 3 Day 1 is at least 80% (i.e., non-inferiority margin of 20%). Non-inferiority in terms of ORR is reached when the lower bound of the 95% CI is ≥60% (i.e., 60% retention of ORR).

“Overall response rate” (ORR) refers to the proportion of subjects who achieve partial response (PR), very good partial response (VGPR), complete response (CR) or stringent complete response (sCR) during or after the treatment.

“Overall survival” (OS) is defined as the time from initiation of therapy to the date of death due to any cause. For the purpose of the clinical trial described in the example, OS is defined as the time from randomization of study population to the date of the patient's death.

“Percent w/v” (% w/v) refers to weight in grams per 100 mL.

“Pharmaceutical combination” refers to a combination of two or more therapeutics or drugs administered either together or separately.

“Pharmaceutical composition” refers to a product that results from combining an antibody that specifically binds CD38 and a hyaluronidase as a fixed combination. “Fixed combinations” refers to a single pharmaceutical composition comprising the anti-CD38 antibody and the hyaluronidase administered simultaneously in the form of a single entity or dosage. A pharmaceutical composition typically includes a pharmaceutically acceptable carrier.

“Pharmaceutically acceptable carrier” or “excipient” refers to an ingredient in a pharmaceutical composition, other than the active ingredient, which is nontoxic to a subject. A pharmaceutically acceptable carrier includes, but is not limited to, a buffer, stabilizer or preservative.

“Platin compound” refers to carboplatin, cisplatin, cisplatinum and oxaliplatin. Carboplatin is marketed under the trade name PARAPLATIN®. Cisplatin is marketed under the trade name PLATINOL®. Oxaliplatin is marketed under the trade name Eloxatin™.

“Post-ASCT and consolidation CR rate” refers to the proportion of subjects who have achieved CR or better by the end of consolidation therapy.

“Post-ASCT and consolidation MRD negative rate” refers to the proportion of subjects who have achieved MRD negative status by the end of consolidation therapy.

“Post-consolidation” refers to treatment period ending at the end of consolidation therapy.

“Post-induction” refers to treatment period ending at the end of induction therapy.

“Post-induction stringent complete response rate” (post-induction sCR rate) refers to the proportion of subjects who have achieved sCR prior to HDC and ASCT.

“Post-induction overall response rate” (post-induction ORR) refers to the proportion of subjects who have achieved partial response (PR) or better by the end of induction.

“Post-induction very good partial response or better” (post-induction VGPR or better) refers to the proportion of subjects who have achieved VGPR, complete response (CR) or stringent complete response (sCR) by the end of induction.

“Progression-free survival” (PFS) means time from initiation of therapy to first evidence of disease progression or death due to any cause, whichever occurs first. For the purpose of the clinical trial described in the example, PFS is defined as the duration from the date of randomization of study population to the first documented progressive disease (PD) or death due to any cause, whichever occurs first.

“Progression-free survival 2” (PFS2) refers to the time from the second randomization to time of subsequent progression on next-line of therapy after disease progression on study treatment.

“Progressive disease” (PD), “stable disease” (SD), “partial response” (PR), “very good partial response” (VGPR), “complete response” (CR) and “stringent complete response” (sCR) refer to response to treatment and take their customary meanings as will be understood by a person skilled in the art of designing, conducting, or reviewing clinical trials. Response to treatment may be assessed using International Myeloma Working Group (IMWG) uniform response criteria recommendations (International Uniform Response Criteria Consensus Recommendations) as shown in Table 1.

“Reducing” in the context of IRR refers to lessening severity or occurrence of IRR observed after subcutaneous administration of the antibody that specifically binds CD38 when compared to severity or occurrence of IRR observed after intravenous administration of the antibody that specifically binds CD38.

“RLD” may also refer to an approved biological product such as DARZALEX® brand of daratumumab against which a biosimilar product is compared.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Safe” as it relates to a composition, dose, dosage regimen, treatment or method with a therapeutic or a drug (such as an antibody that specifically binds CD38 or a combination of an antibody that specifically binds CD38 and one or more therapeutic agents) refers to a favorable benefit:risk ratio with an acceptable frequency and/or acceptable severity of adverse events (AEs) and/or treatment-emergent adverse events (TEAEs) compared to the standard of care or to another comparator.

“Safe and effective” refers to an amount and/or dosage of a drug (such as an antibody that specifically binds CD38) or a combination of drugs (such as a combination of an antibody that specifically binds CD38 and one or more therapeutic agents) that elicits the desired biological or medicinal response in a subject's biological system without the risks outweighing the benefits of such response in accordance with the Federal Food, Drug, and Cosmetic Act, as amended (secs. 201-902, 52 Stat. 1040 et seq., as amended; 21 U.S.C. §§ 321-392). Safety is evaluated in laboratory, animal and human clinical testing to determine the highest tolerable dose or the optimal dose of the drug or the combination of drugs needed to achieve the desired benefit. Efficacy is evaluated in human clinical trials and determining whether the drug or the combination of drugs demonstrates a health benefit over a placebo or other intervention. Safe and effective drugs or a combination of drugs are granted marketing approval by the FDA for their indicated use.

An antibody that “specifically binds CD38” refers to antibody binding CD38 with greater affinity than to other antigens. Typically, the antibody binds to CD38 with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with a K_{D} that is at least one hundred-fold less than its K_{D} for binding to a non-specific antigen (e.g., BSA, casein). The K_{D} may be measured using standard procedures. Antibodies that specifically bind CD38 may, however, have cross-reactivity to other related antigens, for example to the same antigen from other species (homologs), such as monkey, for example Macaca fascicularis (cynomolgus, cyno), Pan troglodytes (chimpanzee, chimp) or Callithrix jacchus (common marmoset, marmoset).

“Stringent complete response rate or better” (sCR rate or better) refers to the proportion of subjects achieving sCR during or after the treatment.

“Subcutaneous” or “subcutaneously” refers to administration of a therapeutic under the skin, typically by injection. Administration site may be side or back of upper arm, front of thigh or abdomen.

“Subject” refers to a human patient. The terms “subject” and “patient” can be used interchangeably herein.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

“Time to disease progression” (TTP) means time from the date of randomization to the date of confirmed progressive disease (PD) or death due to PD, whichever occurs first.

“Time to disease progression 2” (TTP2) refers to the time from the date of second randomization to confirmed progressive disease (PD) or death due to PD, whichever occurs first.

“Time to next treatment” (TNT or TTNT) refers to the time from randomization to the start of the next-line treatment.

“Time to response” refers to the time between the randomization and the first efficacy evaluation that the subject has met all criteria for partial response (PR) or better.

“Time to subsequent anti-myeloma therapy” refers to the time from the initiation of therapy to documentation of administration of a new anti-myeloma therapy to the subject.

“Treat”, “treating” or “treatment” refers to therapeutic treatment. Individuals in need of treatment include those subjects diagnosed with the disorder of a symptom of the disorder. Subject that may be treated also include those prone or susceptible to have the disorder, or those in which the disorder is to be prevented. Beneficial or desired clinical results include alleviation of symptoms, diminishment of extent of disease, stabilized (i.e., not worsening) state of disease, delay or slowing of disease progression, amelioration or palliation of the disease state, disease remission (whether partial or total) and prolonging survival as compared to expected survival if a subject was not receiving treatment or was receiving another treatment.

“Treatment emergent adverse events” (TEAE) as used herein takes its customary meaning as will be understood by a person skilled in the art of designing, conducting, or reviewing clinical trials and the data generated in such trials and refers to an AE considered associated with the use of an antibody that specifically binds CD38 if the attribution is possible, probable, or very likely.

“Unacceptable adverse events” and “unacceptable adverse reaction” refers to all harm or undesired outcomes associated with or caused by administration of a pharmaceutical composition or a therapeutic, and the harm or undesired outcome reaches such a level of severity that a regulatory agency deems the pharmaceutical composition or the therapeutic unacceptable for the proposed use.

“Very good partial response or better” (VGPR rate or better) refers to the proportion of subjects achieving VGPR, complete response (CR) or stringent complete response (sCR) during or after the treatment.

### Multiple Myeloma

Multiple myeloma causes significant morbidity and mortality. It accounts for approximately 1% of all malignancies and 13% of hematologic cancers worldwide. Approximately 50,000 patients per year are diagnosed with multiple myeloma in the EU and US, and 30,000 patients per year die due to multiple myeloma.

The majority of patients with multiple myeloma produce a monoclonal protein (paraprotein, M-protein or M-component) which is an immunoglobulin (Ig) or a fragment of one that has lost its function (Kyle and Rajkumar, Leukemia 23:3-9, 2009; Palumbo and Anderson, N Engl J Med 364:1046-1060, 2011). Normal immunoglobulin levels are compromised in patients, leading to susceptibility of infections. The proliferating multiple myeloma cells displace the normal bone marrow leading to dysfunction in normal hematopoietic tissue and destruction of the normal bone marrow architecture, which is reflected by clinical findings such as anemia, paraprotein in serum or urine, and bone resorption seen as diffuse osteoporosis or lytic lesions shown in radiographs (Kyle et al., Mayo Clin Proc 78:21-33, 2003). Furthermore, hypercalcemia, renal insufficiency or failure, and neurological complications are frequently seen. A small minority of patients with multiple myeloma are non-secretory.

Treatment choices for multiple myeloma vary with age, comorbidity, the aggressiveness of the disease, and related prognostic factors (Palumbo and Anderson, N Engl J Med 364:1046-1060, 2011). Newly diagnosed patients with multiple myeloma are typically categorized into 2 subpopulations usually defined by their age and suitability for the subsequent approach to treatment. Younger patients will typically receive an induction regimen followed by consolidation treatment with high-dose chemotherapy (HDC) and autologous stem cell transplantation (ASCT). For those not considered suitable for HDC and ASCT, longer-term treatment with multi-agent combinations including alkylators, high-dose steroids, and novel agents are currently considered as standards of care. In general, patients over the age of 65 or with significant comorbidities are usually not considered eligible for HDC and ASCT. For many years, the oral combination melphalan-prednisone (MP) was considered the standard of care for patients with multiple myeloma who were not eligible for ASCT (Gay and Palumbo, Blood Reviews 25:65-73, 2011). The advent of immunomodulatory agents (IMiDs) and proteasome inhibitors (PIs) has led to a multiplicity of new treatment options for newly diagnosed patients not considered suitable for transplant-based therapy.

### Multiple Myeloma Diagnosis

Subjects afflicted with multiple myeloma satisfy the CRAB (calcium elevation, renal insufficiency, anemia and bone abnormalities) criteria, and have clonal bone marrow plasma cells ≥10% or biopsy-proven bony or extramedullary plasmacytoma, and measurable disease. Measurable disease is defined by any of the following;
IgG myeloma: Serum monoclonal paraprotein (M-protein) level ≥1.0 g/dL or urine M-protein level ≥200 mg/24 hours; or
IgA, IgM, IgD, or IgE multiple myeloma: serum M-protein level ≥0.5 g/dL or urine M-protein level ≥200 mg/24 hours; or
Light chain multiple myeloma without measurable disease in serum or urine: Serum immunoglobulin free light chain ≥10 mg/dL and abnormal serum immunoglobulin kappa lambda free light chain ratio.

### CRAB Criteria

Hypercalcemia: serum calcium >0.25 mM/L (>1 mg/dL) higher than the upper limit of the normal range [ULN] or >2.75 mM/L (>11 mg/dL)
Renal insufficiency: creatinine clearance <40 mL/min or serum creatinine >177 μM/L (>2 mg/dL)
Anemia: hemoglobin >2 g/dL below the lower limit of normal or hemoglobin <10 g/dL
Bone lesions: one or more osteolytic lesions on skeletal radiography, CT, or PET-CT

Response to treatment may be assessed using International Myeloma Working Group (IMWG) uniform response criteria recommendations (International Uniform Response Criteria Consensus Recommendations) as shown in Table 1.

TABLE 1

| Response | Response Criteria |
|---|---|
| Stringent | CR as defined below, plus |
| complete | Normal FLC ratio, and |
| Response | Absence of clonal PCs by immunohistochemistry, |
| (sCR) | immunofluorescence or 2-to 4-color flow cytometry |
| Complete | Negative immunofixation on the serum and urine, and |
| response (CR) | Disappearance of any soft tissue plasmacytomas, and |
| | <5% PCs in bone marrow |
| Very good | Serum and urine M-component detectable by immunofixation but |
| partial | not on electrophoresis, |
| Response | or |
| (VGPR) | ≥90% reduction in serum M-protein plus urine M-protein |
| | <100 mg/24 hours |
| Partial | ≥50% reduction of serum M-protein and reduction in 24-hour |
| response (PR) | urinary M-protein by ≥90% or to <200 mg/24 hours |
| | If the serum and urine M-protein are not measurable, a decrease of |
| | ≥50% in the difference between involved and uninvolved FLC |
| | levels is required in place of the M-protein criteria |
| | If serum and urine M-protein are not measurable, and serum free |
| | light assay is also not measurable, ≥50% reduction in bone marrow |
| | PCs is required in place of M-protein, provided baseline bone |
| | marrow plasma cell percentage was ≥30% |
| | In addition to the above criteria, if present at baseline, a ≥50% |
| | reduction in the size of soft tissue plasmacytomas is also required. |
| Stable disease | Not meeting criteria for CR, VGPR, PR, or progressive disease |
| (SD) | |
| Progressive | Increase of 25% from lowest response value in any one of the |
| disease (PD) | following: |
| | Serum M-component (absolute increase must be ≥0.5 g/dL), |
| | Urine M-component (absolute increase must be ≥200 mg/24 |
| | hours), |
| | Only in subjects without measurable serum and urine M-protein |
| | levels: the difference between involved and uninvolved FLC levels |
| | (absolute increase must be >10 mg/dL) |
| | Only in subjects without measurable serum and urine M-protein |
| | levels and without measurable disease by FLC levels, bone marrow |
| | PC percentage (absolute percentage must be ≥10%) |
| | Bone marrow plasma cell percentage: the absolute percentage must |
| | be >10% |
| | Definite development of new bone lesions or soft tissue |
| | plasmacytomas or definite increase in the size of existing bone |
| | lesions or soft tissue plasmacytomas |
| | Development of hypercalcemia (corrected serum calcium >11.5 |
| | mg/dL) that can be attributed solely to the PC proliferative disorder |

EBMT = European Group for Blood and Marrow Transplantation; FLC = free light chain; PC = plasma cell

Methods of Treatment and Uses with Clinically Proven Pharmaceutical Compositions

The disclosure provides clinically proven pharmaceutical compositions comprising an antibody that specifically binds CD38 comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and recombinant human hyaluronidase (rHuPH20) which are suitable for subcutaneous administration and treatment methods using the pharmaceutical compositions. The disclosure is based on phase 3 clinical trial results demonstrating non-inferiority of subcutaneously administered pharmaceutical compositions of the disclosure when compared to intravenous administration of DARXALEX® (daratumumab), as well as significantly reduced infusion-related reactions (IRR) accompanied with subcutaneous administration of the pharmaceutical compositions of the disclosure.

As the clinically proven nature and non-inferiority were demonstrated, the pharmaceutical composition comprising the antibody that specifically binds CD38 comprising the HCDR1 of SEQ ID NO:1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20 is interchangeable with DARZALEX® (daratumumab) administered intravenously.

The disclosure provides a method of treating a subject having a disease involving cells expressing CD38 who is expected to benefit from a treatment with an antibody that specifically binds CD38, comprising:
providing a healthcare professional a pharmaceutical composition comprising an antibody that specifically binds CD38 comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6 and recombinant human hyaluronidase (rHuPH20), wherein the pharmaceutical composition is clinically proven for subcutaneous administration;
providing the healthcare professional information that the pharmaceutical composition is clinically proven for subcutaneous administration; wherein performing the steps a) and b) results in the medical professional to administer subcutaneously the pharmaceutical composition to the subject having the disease involving cells expressing CD38, thereby treating the subject having the disease involving cells expressing CD38.

The disclosure also provides a method of reducing occurrence or severity of infusion related reactions (IRR) in a subject who is treated with an antibody that specifically binds CD38, comprising
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration;
providing the healthcare professional information that the pharmaceutical composition is clinically proven for subcutaneous administration and that subcutaneous administration of the pharmaceutical composition has been demonstrated to result in reduced occurrence or severity of IRR; wherein performing the steps a) and b) results in the healthcare professional to administer subcutaneously the pharmaceutical composition to the subject, thereby reducing occurrence or severity of IRR in the subject.

Phase 3 clinical trial results demonstrated that treatment with subcutaneously administered daratumumab reduced any grade IRRs from 34.5% to 12.7%, and Grade 3 IRRs from 5.4% to 1.5% when compared to intravenously administered DARZALEX® (daratumumab).

The disclosure also provides a method of treating a subject having a disease involving cells expressing CD38 who is expected to benefit from a treatment with an antibody that specifically binds CD38, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration.

The disclosure also provides a method of reducing occurrence or severity of infusion related reactions (IRR) in a subject who is treated with an antibody that specifically binds CD38, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration.

In some embodiments, the disease involving cells expressing CD38 is a cancer, an inflammatory disease or an autoimmune disease.

In some embodiments, the cancer is a CD38-positive hematological malignancy.

In some embodiments, the CD38-positive hematological malignancy is multiple myeloma.

In some embodiments, the CD38-positive hematological malignancy is diffuse large B-cell lymphoma (DLBCL).

In some embodiments, the CD38-positive hematological malignancy is non-Hodgkin's lymphoma (NHL).

In some embodiments, the CD38-positive hematological malignancy is acute lymphoblastic leukemia (ALL).

In some embodiments, the CD38-positive hematological malignancy is follicular lymphoma (FL).

In some embodiments, the CD38-positive hematological malignancy is Burkitt's lymphoma (BL).

In some embodiments, the CD38-positive hematological malignancy is mantle cell lymphoma (MCL).

In some embodiments, the CD38-positive hematological malignancy is light chain amyloidosis (AL).

In some embodiments, the CD38-positive hematological malignancy is smoldering multiple myeloma (SMM).

In some embodiments, the CD38-positive hematological malignancy is plasma monoclonal gammopathy of undetermined significance (MGUS).

In some embodiments, the CD38-positive hematological malignancy is multiple myeloma, acute lymphoblastic leukemia (ALL), non-Hodgkin's lymphoma (NHL), diffuse large B-cell lymphoma (DLBCL), Burkitt's lymphoma (BL), follicular lymphoma (FL), mantle-cell lymphoma (MCL), light chain amyloidosis (AL), smoldering multiple myeloma (SMM) or plasma monoclonal gammopathy of undetermined significance (MGUS).

Exemplary B-cell non-Hodgkin's lymphomas are lymphomatoid granulomatosis, primary effusion lymphoma, intravascular large B-cell lymphoma, mediastinal large B-cell lymphoma, heavy chain diseases (including γ, μ, and a disease), lymphomas induced by therapy with immunosuppressive agents, such as cyclosporine-induced lymphoma, and methotrexate-induced lymphoma.

In some embodiments, the cancer is a solid tumor.

In some embodiments, the solid tumor is lung cancer, a non-small cell lung cancer or a small cell lung cancer.

In some embodiments, the autoimmune disease is rheumatoid arthritis or lupus.

The disclosure also provides a method of treating a subject with multiple myeloma, comprising:
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration;
providing the healthcare professional information that the pharmaceutical composition is clinically proven for subcutaneous administration; wherein performing the steps a) and b) results in the medical professional to administer subcutaneously the pharmaceutical composition to the subject having multiple myeloma, thereby treating the subject having multiple myeloma.

The disclosure also provides a method of treating a subject with multiple myeloma, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is clinically proven for subcutaneous administration.

In some embodiments, the method demonstrates non-inferiority to intravenous administration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6.

In some embodiments, the method demonstrates non-inferiority to intravenous administration of DARZALEX® (daratumumab).

In some embodiments, non-inferiority is demonstrated using overall response rate (ORR).

In some embodiments, non-inferiority is demonstrated using maximum C_{trough} concentration.

Maximum C_{trough} concentration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 may be assessed from serum samples using known methods such as ELISA.

In some embodiments, the pharmaceutical composition comprises about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 30,000 U rHuPH20.

In some embodiments, the pharmaceutical composition comprises about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 2,000 U/mL rHuPH20.

In some embodiments, the pharmaceutical composition comprises one or more excipients.

In some embodiments, the one or more excipients is histidine, methionine, sorbitol or polysorbate-20 (PS-20), or any combination thereof.

In some embodiments, the pharmaceutical composition comprises
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical composition comprises about 10 mM histidine.

In some embodiments, the pharmaceutical composition comprises about 300 mM sorbitol.

In some embodiments, the pharmaceutical composition comprises about 0.04% (w/v) PS-20.

In some embodiments, the pharmaceutical composition comprises about 1 mg/mL methionine.

In some embodiments, the pharmaceutical composition comprises
about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 2,000 U/mL of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 500 U/mL of rHuPH20;
about 10 mM histidine;
about 300 mM sorbitol;
about 0.04% w/v PS-20; and
about 2 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 500 U/mL of rHuPH20;
about 10 mM histidine;
about 300 mM sorbitol; and
about 2 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 500 U/mL of rHuPH20;
about 10 mM histidine;
about 300 mM sorbitol;
about 0.01% w/v PS-20; and
about 2 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 500 U/mL of rHuPH20;
about 10 mM histidine;
about 300 mM sorbitol;
about 0.02% w/v PS-20; and
about 2 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 500 U/mL of rHuPH20;
about 10 mM histidine;
about 300 mM sorbitol;
about 0.06% w/v PS-20; and
about 2 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 50 U/mL of rHuPH20;
about 10 mM histidine;
about 300 mM sorbitol;
about 0.04% w/v PS-20; and
about 1 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 500 U/mL of rHuPH20;
about 10 mM histidine;
about 300 mM sorbitol;
about 0.04% w/v PS-20; and
about 1 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 2,000 U/mL of rHuPH20;
about 10 mM histidine;
about 300 mM sorbitol;
about 0.04% w/v PS-20; and
about 1 mg/mL methionine; at pH about 5.5.

In some embodiments, the pharmaceutical composition comprises
about 100 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 5,000 U/mL of rHuPH20;
about 10 mM histidine;
about 300 mM sorbitol;
about 0.04% w/v PS-20; and
about 1 mg/mL methionine; at pH about 5.5.

The pharmaceutical composition of the disclosure may alternatively comprise additional or alternative pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Exemplary buffers that may be used are acetic acid, citric acid, formic acid, succinic acid, phosphoric acid, carbonic acid, malic acid, aspartic acid, histidine, boric acid, Tris buffers, HEPPSO and HEPES.

Exemplary antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Exemplary amino acids that may be used are histidine, isoleucine, methionine, glycine, arginine, lysine, L-leucine, tri-leucine, alanine, glutamic acid, L-threonine, and 2-phenylamine.

Exemplary surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the Monaqua™ series (Mona Industries, Inc., Paterson, N.J.), polyethyl glycol, polypropyl glycol, and copolymers of ethylene and propylene glycol (e.g., Pluronics™, PF68, etc).

Exemplary preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Exemplary saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Exemplary salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. An exemplary salt is sodium chloride.

In some embodiments, the pharmaceutical composition of the disclosure comprises saccharide.

In some embodiments, saccharide is sucrose.

In some embodiments, saccharide is sorbitol.

In some embodiments, saccharide is mannitol.

In some embodiments, the pharmaceutical composition of the disclosure comprises polysorbate.

In some embodiments, the pharmaceutical composition of the disclosure comprises polysorbate-20 (PS-20).

In some embodiments, the pharmaceutical composition of the disclosure comprises polysorbate-20 (PS-20) at a concentration of from about 0.01% w/v to about 0.1% w/v.

In some embodiments, the pharmaceutical composition of the disclosure comprises polysorbate-20 (PS-20) at a concentration of from about 0.01% w/v to about 0.08% w/v.

In some embodiments, the pharmaceutical composition of the disclosure comprises polysorbate-20 (PS-20) at a concentration of from about 0.01% w/v to about 0.04% w/v.

In some embodiments, the pharmaceutical composition of the disclosure comprises polysorbate-20 (PS-20) at a concentration of about 0.01% w/v, 0.02% w/v, 0.03% w/v, 0.04% w/v, 0.05% w/v, 0.06% w/v, 0.07% w/v, 0.08% w/v, 0.09% w/v or 0.1% w/v.

In some embodiments, the pharmaceutical composition of the disclosure comprises histidine.

In some embodiments, the pharmaceutical composition of the disclosure comprises histidine at a concentration of from about 1 mM to about 50 mM.

In some embodiments, the pharmaceutical composition of the disclosure comprises histidine at a concentration of from about 5 mM to about 50 mM.

In some embodiments, the pharmaceutical composition of the disclosure comprises histidine at a concentration of from about 5 mM to about 30 mM.

In some embodiments, the pharmaceutical composition of the disclosure comprises histidine at a concentration of from about 5 mM to about 20 mM.

In some embodiments, the pharmaceutical composition of the disclosure comprises histidine at a concentration of from about 5 mM to about 15 mM.

In some embodiments, the pharmaceutical composition of the disclosure comprises histidine at a concentration of from about 5 mM to about 10 mM.

In some embodiments, the pharmaceutical composition of the disclosure comprises histidine at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM or about 50 mM.

In some embodiments, the pharmaceutical composition of the disclosure comprises histidine at a concentration of about 5 mM.

In some embodiments, the pharmaceutical composition of the disclosure comprises histidine at a concentration of about 10 mM.

In some embodiments, the pharmaceutical composition of the disclosure comprises histidine at a concentration of about 15 mM.

In some embodiments, the pharmaceutical composition of the disclosure comprises histidine at a concentration of about 20 mM.

In some embodiments, the pharmaceutical composition of the disclosure comprises sorbitol at a concentration of from about 50 mM to about 500 mM.

In some embodiments, the pharmaceutical composition of the disclosure comprises sorbitol at a concentration of from about 50 mM to about 450 mM.

In some embodiments, the pharmaceutical composition of the disclosure comprises sorbitol at a concentration of from about 50 mM to about 400 mM.

In some embodiments, the pharmaceutical composition of the disclosure comprises sorbitol at a concentration of from about 50 mM to about 350 mM.

In some embodiments, the pharmaceutical composition of the disclosure comprises sorbitol at a concentration of from about 100 mM to about 350 mM.

In some embodiments, the pharmaceutical composition of the disclosure comprises sorbitol at a concentration of from about 100 mM to about 300 mM.

In some embodiments, the pharmaceutical composition of the disclosure comprises sorbitol at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition of the disclosure comprises sorbitol at a concentration of about 50 mM.

In some embodiments, the pharmaceutical composition comprises sorbitol at a concentration of about 100 mM.

In some embodiments, the pharmaceutical composition comprises sorbitol at a concentration of about 150 mM.

In some embodiments, the pharmaceutical composition comprises sorbitol at a concentration of about 200 mM.

In some embodiments, the pharmaceutical composition comprises sorbitol at a concentration of about 250 mM.

In some embodiments, the pharmaceutical composition comprises sorbitol at a concentration of about 300 mM.

In some embodiments, the pharmaceutical composition comprises sorbitol at a concentration of about 350 mM.

In some embodiments, the pharmaceutical composition comprises sorbitol at a concentration of about 400 mM.

In some embodiments, the pharmaceutical composition comprises sucrose at a concentration of from about 50 mM to about 500 mM.

In some embodiments, the pharmaceutical composition comprises sucrose at a concentration of from about 50 mM to about 450 mM.

In some embodiments, the pharmaceutical composition comprises sucrose at a concentration of from about 50 mM to about 400 mM.

In some embodiments, the pharmaceutical composition comprises sucrose at a concentration of from about 50 mM to about 350 mM.

In some embodiments, the pharmaceutical composition comprises sucrose at a concentration of from about 100 mM to about 350 mM.

In some embodiments, the pharmaceutical composition comprises sucrose at a concentration of from about 100 mM to about 200 mM.

In some embodiments, the pharmaceutical composition comprises sucrose at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition comprises sucrose at a concentration of about 50 mM.

In some embodiments, the pharmaceutical composition comprises sucrose at a concentration of about 100 mM.

In some embodiments, the pharmaceutical composition comprises sucrose at a concentration of about 150 mM.

In some embodiments, the pharmaceutical composition comprises sucrose at a concentration of about 200 mM.

In some embodiments, the pharmaceutical composition comprises sucrose at a concentration of about 250 mM.

In some embodiments, the pharmaceutical composition comprises sucrose at a concentration of about 300 mM.

In some embodiments, the pharmaceutical composition comprises sucrose at a concentration of about 350 mM.

In some embodiments, the pharmaceutical composition comprises sucrose at a concentration of about 400 mM.

In some embodiments, the pharmaceutical composition comprises methionine.

In some embodiments, the pharmaceutical composition comprises methionine at a concentration of from about 0.1 mg/mL to about 5 mg/mL.

In some embodiments, the pharmaceutical composition comprises methionine at a concentration of from about 0.1 mg/mL to about 2.5 mg/mL.

In some embodiments, the pharmaceutical composition comprises methionine at a concentration of from about 1 mg/mL to about 2 mg/mL.

In some embodiments, the pharmaceutical composition comprises methionine at a concentration of about 0.5 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1/7 mg/mL, about 1.8 mg/mL, about 1.9 mg/mL, about 2.0 mg/mL, about 2.1 mg/mL, about 2.2 mg/mL, about ⅔ mg/mL, about 2.4 mg/mL, about 2.5 mg/mL, about 2.6 mg/mL, about 2.7 mg/mL, about 2.8 mg/mL, about 2.9 mg/mL, about 3 mg/mL, about 3.5 mg/mL, about 4 mg/mL, about 4.5 mg/mL or about 5 mg/mL.

In some embodiments, the pharmaceutical composition is at pH 5.0 to 6.0.

In some embodiments, the pharmaceutical composition is at pH 5.3 to 5.8.

In some embodiments, the pharmaceutical composition is at pH 5.5.

In some embodiments, the pharmaceutical composition is at pH 5.6.

In some embodiments, the antibody that specifically binds CD38 comprises a heavy chain variable region (VH) of SEQ ID NO: 7 and a light chain variable region (VL) of SEQ ID NO: 8.

In some embodiments, the antibody that specifically binds CD38 is an IgG1 isotype.

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as G1m17, G1m3, G1m1, G1m2, G1m27 or G1m28.

In some embodiments, the antibody that specifically binds CD38 comprises a heavy chain (HC) of SEQ ID NO: 9 and a light chain (LC) of SEQ ID NO: 10.

In some embodiments, the antibody that specifically binds CD38 is daratumumab.

In some embodiments, the antibody that specifically binds CD38 is a biosimilar of DARZALEX® brand of daratumumab.

In some embodiments, the pharmaceutical composition comprising the antibody that specifically binds CD38 and rHuPH20 is administered at a dose of about 1,800 mg once a week, about 1,800 mg once in two weeks, about 1,800 mg once in three weeks or about 1,800 mg once in four weeks.

In some embodiments, the pharmaceutical composition is administered for one or more 28-day cycles.

In some embodiments, the pharmaceutical composition is administered once a week in the first and the second 28-day cycle, once in two weeks in the third and the fourth 28-day cycle, and thereafter once in four weeks in any subsequent 28-day cycle.

In some embodiments, the pharmaceutical composition is administered in combination with one or more additional therapeutics.

In some embodiments, the one or more additional therapeutics is an immunomodulatory agent, a corticosteroid, a chemotherapeutic agent, an antineoplastic antimetabolite, a platin compound or high dose chemotherapy (HDC) and stem cell transplant (SCT).

In some embodiments, the immunomodulatory agent is a glutamic acid derivative.

In some embodiments, the glutamic acid derivative is lenalinomide, pomalidomide or thalidomide, or any combination thereof.

In some embodiments, the corticosteroid is dexamethasone or prednisone, or any combination thereof.

In some embodiments, the chemotherapeutic agent is a proteasome inhibitor.

In some embodiments, the proteasome inhibitor is bortezomib, carfilzomib, marizomib or ixazomib, or any combination thereof.

In some embodiments, the chemotherapeutic agent is an alkylating agent.

In some embodiments, the alkylating agent is melphalan, cyclophosphamide, ifosfamide or nitrosourea, or any combination thereof.

In some embodiments, the chemotherapeutic agent is a microtubule inhibitor (MTI).

In some embodiments, the MTI is a taxane or a vinca alkaloid, or any combination thereof.

In some embodiments, the vinca alkaloid is vincristine.

In some embodiments, SCT is autologous SCT (ASCT), allogenic SCT or syngeneic SCT.

In some embodiments, SCT is ASCT.

In some embodiments, the one or more additional therapeutics comprises bortezomib and dexamethasone.

In some embodiments, bortezomib is administered at a dose of about 1.3 mg/m² and dexamethasone is administered at a dose of about 20 mg.

In some embodiments, the one or more additional therapeutics comprises lenalidomide and dexamethasone.

In some embodiments, lenalidomide is administered at a dose of about 25 mg and dexamethasone is administered at a dose of between about 20 mg and about 40 mg.

In some embodiments, the one or more additional therapeutics comprises pomalidomide and dexamethasone.

In some embodiments, pomalidomide is administered at a dose of about 25 mg and dexamethasone is administered at a dose of between about 20 mg and about 40 mg.

In some embodiments, the one or more additional therapeutics comprises bortezomib, melphalan and prednisone.

In some embodiments, bortezomib is administered at a dose of about 1.3 mg/m², melphalan is administered at a dose of about 9 mg/m² and prednisone is administered at a dose of about 60 mg/m².

In some embodiments, the one or more additional therapeutics comprises bortezomib, thalidomide and dexamethasone.

In some embodiments, bortezomib is administered at a dose of about 1.3 mg/m², thalidomide is administered at a dose of about 25 mg and dexamethasone is administered at a dose of about between about 20 mg and about 40 mg.

In some embodiments, multiple myeloma is relapsed, refractory, or both relapsed and refractory.

In some embodiments, multiple myeloma is newly diagnosed multiple myeloma.

In some embodiments, the subject is eligible for high dose chemotherapy (HDC) and stem cell transplant (SCT).

In some embodiments, SCT is autologous SCT (ASCT), allogenic SCT or syngeneic SCT.

In some embodiments, SCT is ASCT.

In some embodiments, HDC is melphalan.

### Clinically Proven Pharmaceutical Compositions

The disclosure also provides a pharmaceutical composition comprising a clinically proven amount of an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20, wherein the pharmaceutical composition is intended for subcutaneous administration.

The disclosure also provides a pharmaceutical composition for subcutaneous administration comprising a clinically proven amount of an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20.

In some embodiments, the antibody that specifically binds CD38 comprises the VH of SEQ ID NO: 7 and the VL of SEQ ID NO: 8.

In some embodiments, the antibody that specifically binds CD38 is an IgG1 isotype.

In some embodiments, the antibody that specifically binds CD38 comprises the HC of SEQ ID NO: 9 and the LC of SEQ ID NO: 10.

In some embodiments, the pharmaceutical composition comprises about 1,800 mg of the antibody that specifically binds CD38 and about 30,000 U rHuPH20.

In some embodiments, the pharmaceutical composition comprises about 120 mg/mL of the antibody that specifically binds CD38 and about 2,000 U/mL rHuPH20.

In some embodiments, the pharmaceutical composition further comprises one or more excipients.

In some embodiments, the one or more excipients is histidine, methionine, sorbitol or polysorbate-20 (PS-20), or any combination thereof.

In some embodiments, the pharmaceutical composition comprises
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical composition comprises about 10 mM histidine.

In some embodiments, the pharmaceutical composition comprises about 300 mM sorbitol.

In some embodiments, the pharmaceutical composition comprises about 0.04% (w/v) PS-20.

In some embodiments, the pharmaceutical composition comprises about 1 mg/mL methionine.

In some embodiments, the pharmaceutical composition comprises
about 1,800 mg of the antibody that specifically binds CD38;
about 30,000 U or rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 120 mg/mL of the antibody that specifically binds CD38;
about 2,000 U/mL or rHuPH20;
about 10 mM histidine;
about 300 mM sorbitol;
about 0.04% (w/v) PS-20; and
about 1 mg/mL methionine, at a pH of about 5.6.

In some embodiments, subcutaneous administration of the pharmaceutical composition to a subject results in reduced occurrence or severity of infusion related reactions (IRR) in a subject when compared to intravenous administration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6.

### Administration

The pharmaceutical composition of the disclosure is administered by subcutaneous administration.

The pharmaceutical composition of the disclosure may is be administered in a total volume of about 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 105 mL, 110 mL, 115 mL or 120 mL.

In some embodiments the pharmaceutical composition of the disclosure is administered in a total volume of about 10 mL.

In some embodiments, the pharmaceutical composition of the disclosure is administered in a total volume of about 11 mL.

In some embodiments, the pharmaceutical composition of the disclosure is administered in a total volume of about 12 mL.

In some embodiments, the pharmaceutical composition of the disclosure is administered in a total volume of about 13 mL.

In some embodiments, the pharmaceutical composition of the disclosure is administered in a total volume of about 14 mL.

In some embodiments, the pharmaceutical composition of the disclosure is administered in a total volume of about 15 mL.

In some embodiments, the pharmaceutical composition of the disclosure is administered in a total volume of about 16 mL.

In some embodiments, the pharmaceutical composition of the disclosure is administered in a total volume of about 17 mL.

In some embodiments, the pharmaceutical composition of the disclosure is administered in a total volume of about 18 mL.

In some embodiments, the pharmaceutical composition of the disclosure is administered in a total volume of about 19 mL.

In some embodiments, the pharmaceutical composition of the disclosure is administered in a total volume of about 20 mL.

In some embodiments, the pharmaceutical composition of the disclosure is administered subcutaneously to the abdominal region.

Subcutaneous administration may be accomplished using a device. The device may be a syringe, a prefilled syringe, an auto-injector, either disposable or reusable, a pen injector, a patch injector, a wearable injector or an ambulatory syringe infusion pump with subcutaneous infusion sets.

The pharmaceutical composition of the disclosure may be administered over a time period of between about 1 minute (min) to about 60 min.

In some embodiments, the pharmaceutical composition of the disclosure is administered over the time period of about 1 min.

In some embodiments, the pharmaceutical composition of the disclosure is administered over the time period of about 2 min.

In some embodiments, the pharmaceutical composition of the disclosure is administered over the time period of about 3 min.

In some embodiments, the pharmaceutical composition of the disclosure is administered over the time period of about 4 min.

In some embodiments, the pharmaceutical composition of the disclosure is administered over the time period of about 5 min.

In some embodiments, the pharmaceutical composition of the disclosure is administered over the time period of about 6 min.

In some embodiments, the pharmaceutical composition of the disclosure is administered over the time period of about 7 min.

In some embodiments, the pharmaceutical composition of the disclosure is administered over the time period of about 8 min.

In some embodiments, the pharmaceutical composition of the disclosure is administered over the time period of about 9 min.

In some embodiments, the pharmaceutical composition of the disclosure is administered over the time period of about 10 min.

In some embodiments, the pharmaceutical composition of the disclosure is administered over the time period of about 11 min.

In some embodiments, the pharmaceutical composition of the disclosure is administered over the time period of about 12 min.

In some embodiments, the pharmaceutical composition of the disclosure is administered over the time period of about 13 min.

In some embodiments, the pharmaceutical composition of the disclosure is administered over the time period of about 14 min.

In some embodiments, the pharmaceutical composition of the disclosure is administered over the time period of about 15 min.

In some embodiments, the pharmaceutical composition of the disclosure is administered over the time period of about 16 min.

In some embodiments, the pharmaceutical composition of the disclosure is administered over the time period of about 17 min.

In some embodiments, the pharmaceutical composition of the disclosure is administered over the time period of about 18 min.

In some embodiments, the pharmaceutical composition of the disclosure is administered over the time period of about 19 min.

In some embodiments, the pharmaceutical composition of the disclosure is administered over the time period of about 20 min.

In some embodiments, the pharmaceutical composition of the disclosure is administered over the time period of about 25 min.

In some embodiments, the pharmaceutical composition of the disclosure is administered over the time period of about 30 min.

In some embodiments, the pharmaceutical composition of the disclosure is administered over the time period of about 35 min.

In some embodiments, the pharmaceutical composition of the disclosure is administered over the time period of about 40 min.

In some embodiments, the pharmaceutical composition of the disclosure is administered over the time period of about 45 min.

In some embodiments, the pharmaceutical composition of the disclosure is administered over the time period of about 50 min.

In some embodiments, the pharmaceutical composition of the disclosure is administered over the time period of about 55 min.

In some embodiments, the pharmaceutical composition of the disclosure is administered over the time period of about 60 min.

### Methods of Producing Antibodies

Methods of producing antibodies at large scales are known. Antibodies may be produced for example in CHO cells cultured using known methods. The antibody may be isolated and/or purified from culture medium by removing solids by centrifugation or filtering as a first step in the purification process. The antibody may be further purified by standard methods including chromatography (e.g., ion exchange, affinity, size exclusion, and hydroxyapatite chromatography), gel filtration, centrifugation, or differential solubility, ethanol precipitation or by any other available technique for the purification of antibodies. Protease inhibitors such as phenyl methyl sulfonyl fluoride (PMSF), leupeptin, pepstatin or aprotinin can be added at any or all stages in order to reduce or eliminate degradation of the antibody during the purification process. One of ordinary skill in the art will appreciate that the exact purification technique will vary depending on the character of the polypeptide or protein to be purified, the character of the cells from which the polypeptide or protein is expressed, and the composition of the medium in which the cells were grown.

### Combination Therapies

### Combinations of Clinically Proven Amount of the Pharmaceutical Composition of the Disclosure, Lenalidomide and Dexamethasone

The disclosure also provides a method of treating a subject with multiple myeloma, comprising:
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and recombinant human hyaluronidase (rHuPH20), wherein the pharmaceutical composition is clinically proven for subcutaneous administration;
providing the healthcare professional information that the pharmaceutical composition is clinically proven for subcutaneous administration;
providing the healthcare professional information that the pharmaceutical composition can be administered in combination with lenalidomide and dexamethasone; wherein performing the steps a), b) and c) results in the medical professional to administer subcutaneously the pharmaceutical composition, lenalidomide and dexamethasone to the subject having multiple myeloma, thereby treating the subject having multiple myeloma.

The disclosure also provides a method of treating a subject with multiple myeloma, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20 in combination with lenalidomide and dexamethasone, wherein the pharmaceutical composition is clinically proven for subcutaneous administration.

In some embodiments, the method results in reduced occurrence or severity of infusion related reactions (IRR) in a subject when compared to an intravenous administration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6.

In some embodiments, the pharmaceutical composition comprises about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 30,000 U of rHuPH20.

In some embodiments, the pharmaceutical composition comprises about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 2,000 U/mL of rHuPH20.

In some embodiments, the pharmaceutical composition comprises one or more excipients.

In some embodiments, the one or more excipients is histidine, methionine, sorbitol or polysorbate-20 (PS-20), or any combination thereof.

In some embodiments, the pharmaceutical composition comprises
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, pharmaceutical composition comprises about 10 mM histidine.

In some embodiments, the pharmaceutical composition comprises about 300 mM sorbitol.

In some embodiments, the pharmaceutical composition comprises about 0.04% (w/v) PS-20.

In some embodiments, the pharmaceutical composition comprises about mg/mL methionine.

In some embodiments, the pharmaceutical composition comprises
about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 2,000 U/mL of rHuPH20;
about 10 mM histidine;
about 300 mM sorbitol;
about 0.04% (w/v) PS-20; and
about 1 mg/mL methionine, at a pH of about 5.6.

In some embodiments, the antibody that specifically binds CD38 comprises a heavy chain variable region (VH) of SEQ ID NO: 7 and a light chain variable region (VL) of SEQ ID NO: 8.

In some embodiments, the antibody that specifically binds CD38 is an IgG1 isotype.

In some embodiments, the antibody that specifically binds CD38 comprises a heavy chain (HC) of SEQ ID NO: 9 and a light chain (LC) of SEQ ID NO: 10.

In some embodiments, the antibody that specifically binds CD38 is daratumumab.

In some embodiments, the antibody that specifically binds CD38 is a biosimilar of DARZALEX® brand of daratumumab.

In some embodiments, the pharmaceutical composition comprising the antibody that specifically binds CD38 and rHuPH20 is administered at a dose of about 1,800 mg of the antibody that specifically binds CD38 and about 30,000 U of rHuPH20 once a week, once in two weeks, once in three weeks or once in four weeks.

In some embodiments, lenalidomide is administered at a dose of about 25 mg daily.

In some embodiments, dexamethasone is administered at a dose of between about 20 mg and about 40 mg weekly.

In some embodiments, the pharmaceutical composition, lenalidomide and dexamethasone are administered for one or more 28-day cycles.

In some embodiments, the pharmaceutical composition is administered once a week in the first and the second 28-day cycle, once in two weeks in the third, the fourth, the fifth and the sixth 28-day cycle, and thereafter once in four weeks in any subsequent 28-day cycle.

In some embodiments, lenalidomide is administered daily on days 1-21 in each 28-day cycle.

In some embodiments, dexamethasone is administered at a dose of 20 mg as a pre-infusion medication on the same day when the pharmaceutical composition is administered and optionally at a dose of 20 mg the day after the pharmaceutical composition is administered.

In some embodiments, lenalidomide is administered orally.

In some embodiments, dexamethasone is administered orally or intravenously.

In some embodiments, lenalidomide is self-administered.

In some embodiments, dexamethasone is self-administered.

In some embodiments, multiple myeloma is relapsed, refractory, or both relapsed and refractory.

In some embodiments, multiple myeloma is newly diagnosed multiple myeloma.

In some embodiments, the subject is eligible for high dose chemotherapy (HDC) and stem cell transplant (SCT).

In some embodiments, SCT is autologous SCT (ASCT), allogenic SCT or syngeneic SCT.

In some embodiments, SCT is ASCT.

In some embodiments, HDC is melphalan.

### Combination of Clinically Proven Amount of the Pharmaceutical Composition of the Disclosure, Bortezomib and Dexamethasone

The disclosure also provides a method of treating a subject with multiple myeloma, comprising:
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and recombinant human hyaluronidase (rHuPH20), wherein the pharmaceutical composition is clinically proven for subcutaneous administration;
providing the healthcare professional information that the pharmaceutical composition is clinically proven for subcutaneous administration;
providing the healthcare professional information that the pharmaceutical composition can be administered in combination with bortezomib and dexamethasone; wherein performing the steps a), b) and c) results in the medical professional to administer subcutaneously the pharmaceutical composition, bortezomib and dexamethasone to the subject having multiple myeloma, thereby treating the subject having multiple myeloma.

The disclosure also provides a method of treating a subject with multiple myeloma, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20 in combination with bortezomib and dexamethasone, wherein the pharmaceutical composition is clinically proven for subcutaneous administration.

In some embodiments, the method results in reduced occurrence or severity of infusion related reactions (IRR) in a subject when compared to an intravenous administration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6.

In some embodiments, the pharmaceutical composition comprises about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 30,000 U of rHuPH20.

In some embodiments, the pharmaceutical composition comprises about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 2,000 U/mL of rHuPH20.

In some embodiments, the pharmaceutical composition comprises one or more excipients.

In some embodiments, the one or more excipients is histidine, methionine, sorbitol or polysorbate-20 (PS-20), or any combination thereof.

In some embodiments, the pharmaceutical composition comprises
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical composition comprises about 10 mM histidine.

In some embodiments, the pharmaceutical composition comprises about 300 mM sorbitol.

In some embodiments, the pharmaceutical composition comprises about 0.04% (w/v) PS-20.

In some embodiments, the pharmaceutical composition comprises about mg/mL methionine.

In some embodiments, the pharmaceutical composition comprises
about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 2,000 U/mL of rHuPH20;
about 10 mM histidine;
about 300 mM sorbitol;
about 0.04% (w/v) PS-20; and
about 1 mg/mL methionine, at a pH of about 5.6.

In some embodiments, the antibody that specifically binds CD38 comprises a heavy chain variable region (VH) of SEQ ID NO: 7 and a light chain variable region (VL) of SEQ ID NO: 8.

In some embodiments, the antibody that specifically binds CD38 is an IgG1 isotype.

In some embodiments, the antibody that specifically binds CD38 comprises a heavy chain (HC) of SEQ ID NO: 9 and a light chain (LC) of SEQ ID NO: 10.

In some embodiments, the antibody that specifically binds CD38 is daratumumab.

In some embodiments, the antibody that specifically binds CD38 is a biosimilar of DARZALEX® brand of daratumumab.

In some embodiments, the pharmaceutical composition comprising the antibody that specifically binds CD38 and rHuPH20 is administered at a dose of about 1,800 mg of the antibody that specifically binds CD38 and about 30,000 U of rHuPH20 once a week, once in two weeks, once in three weeks or once in four weeks.

In some embodiments, bortezomib is administered at a dose of about 1.3 mg/m² twice a week.

In some embodiments, dexamethasone is administered at a dose of between about 20 mg and about 80 mg weekly.

In some embodiments, the pharmaceutical composition, bortezomib and dexamethasone is administered for one or more 3-week cycles.

In some embodiments, the pharmaceutical composition is administered once a week in the first, the second and the third 3-week cycle, once in three weeks in the fourth, the fifth, the sixth, the seventh and the eighth 3-week cycle, and thereafter once in four weeks.

In some embodiments, bortezomib is administered at a dose of about 1.3 mg/m² twice a week on week 1 and week 2 in cycles 1-8.

In some embodiments, dexamethasone is administered at a dose of 20 mg as a pre-infusion medication on the same day when the pharmaceutical composition is administered and optionally at a dose of 20 mg the day after the pharmaceutical composition is administered.

In some embodiments, bortezomib is administered subcutaneously or intravenously.

In some embodiments, dexamethasone is administered orally.

In some embodiments, dexamethasone is self-administered.

In some embodiments, multiple myeloma is relapsed, refractory, or both relapsed and refractory.

In some embodiments, multiple myeloma is newly diagnosed multiple myeloma.

In some embodiments, the subject is eligible for high dose chemotherapy (HDC) and stem cell transplant (SCT).

In some embodiments, SCT is autologous SCT (ASCT), allogenic SCT or syngeneic SCT.

In some embodiments, SCT is ASCT.

In some embodiments, HDC is melphalan.

### Combinations of Clinically Proven Amount of the Pharmaceutical Composition of the Disclosure, Pomalidomide and Dexamethasone

The invention also provides a method of treating a subject with multiple myeloma, comprising:
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and recombinant human hyaluronidase (rHuPH20), wherein the pharmaceutical composition is clinically proven for subcutaneous administration;
providing the healthcare professional information that the pharmaceutical composition is clinically proven for subcutaneous administration;
providing the healthcare professional information that the pharmaceutical composition can be administered in combination with pomalidomide and dexamethasone; wherein performing the steps a), b) and c) results in the medical professional to administer subcutaneously the pharmaceutical composition, pomalidomide and dexamethasone to the subject having multiple myeloma, thereby treating the subject having multiple myeloma.

The invention also provides a method of treating a subject with multiple myeloma, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20 in combination with pomalidomide and dexamethasone, wherein the pharmaceutical composition is clinically proven for subcutaneous administration.

In some embodiments, the method results in reduced occurrence or severity of infusion related reactions (IRR) in a subject when compared to an intravenous administration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6.

In some embodiments, the pharmaceutical composition comprises about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 30,000 U of rHuPH20.

In some embodiments, the pharmaceutical composition comprises about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 2,000 U/mL of rHuPH20.

In some embodiments, the pharmaceutical composition comprises one or more excipients.

In some embodiments, the one or more excipients is histidine, methionine, sorbitol or polysorbate-20 (PS-20), or any combination thereof.

In some embodiments, the pharmaceutical composition comprises
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical composition comprises about 10 mM histidine.

In some embodiments, the pharmaceutical composition comprises about 300 mM sorbitol.

In some embodiments, the pharmaceutical composition comprises about 0.04% (w/v) PS-20.

In some embodiments, the pharmaceutical composition comprises about mg/mL methionine.

In some embodiments, the pharmaceutical composition comprises
about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 2,000 U/mL of rHuPH20;
about 10 mM histidine;
about 300 mM sorbitol;
about 0.04% (w/v) PS-20; and
about 1 mg/mL methionine, at a pH of about 5.6.

In some embodiments, the antibody that specifically binds CD38 comprises a heavy chain variable region (VH) of SEQ ID NO: 7 and a light chain variable region (VL) of SEQ ID NO: 8.

In some embodiments, the antibody that specifically binds CD38 is an IgG1 isotype.

In some embodiments, the antibody that specifically binds CD38 comprises a heavy chain (HC) of SEQ ID NO: 9 and a light chain (LC) of SEQ ID NO: 10.

In some embodiments, the antibody that specifically binds CD38 is daratumumab.

In some embodiments, the antibody that specifically binds CD38 is a biosimilar of DARZALEX® brand of daratumumab.

In some embodiments, the pharmaceutical composition comprising the antibody that specifically binds CD38 and rHuPH20 is administered at a dose of about 1,800 mg of the antibody that specifically binds CD38 and about 30,000 U of rHuPH20 once a week, once in two weeks, once in three weeks or once in four weeks.

In some embodiments, pomalidomide is administered at a dose of about 4 mg daily.

In some embodiments, dexamethasone is administered at a dose of between about 20 mg and about 40 mg weekly.

In some embodiments, the pharmaceutical composition, pomalidomide and dexamethasone are administered for one or more 28-day cycles.

In some embodiments, the pharmaceutical composition is administered once a week in the first and the second 28-day cycle, once in two weeks in the third, the fourth, the fifth and the sixth 28-day cycle, and thereafter once in four weeks in any subsequent 28-day cycle.

In some embodiments, pomalidomide is administered daily on days 1-21 in each 28-day cycle.

In some embodiments, dexamethasone is administered at a dose of 20 mg as a pre-infusion medication on the same day when the pharmaceutical composition is administered and optionally at a dose of 20 mg the day after the pharmaceutical composition is administered.

In some embodiments, pomalidomide is administered orally.

In some embodiments, dexamethasone is administered orally or intravenously.

In some embodiments, pomalidomide is self-administered.

In some embodiments, dexamethasone is self-administered.

In some embodiments, multiple myeloma is relapsed, refractory, or both relapsed and refractory.

In some embodiments, myeloma is newly diagnosed multiple myeloma.

In some embodiments, the subject is eligible for high dose chemotherapy (HDC) and stem cell transplant (SCT).

In some embodiments, SCT is autologous SCT (ASCT), allogenic SCT or syngeneic SCT.

In some embodiments, SCT is ASCT.

In some embodiments, HDC is melphalan.

### Combinations of Clinically Proven Amount of the Pharmaceutical Composition of the Disclosure, Bortezomib, Melphalan and Prednisone

The disclosure also provides a method of treating a subject with multiple myeloma, comprising:
providing a healthcare professional a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and recombinant human hyaluronidase (rHuPH20), wherein the pharmaceutical composition is clinically proven for subcutaneous administration;
providing the healthcare professional information that the pharmaceutical composition is clinically proven for subcutaneous administration;
providing the healthcare professional information that the pharmaceutical composition can be administered in combination with bortezomib, melphalan and prednisone; wherein performing the steps a), b) and c) results in the medical professional to administer subcutaneously the pharmaceutical composition, bortezomib, melphalan and prednisone to the subject having multiple myeloma, thereby treating the subject having multiple myeloma.

The disclosure also provides a method of treating a subject with multiple myeloma, comprising subcutaneously administering to the subject a pharmaceutical composition comprising an antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and rHuPH20 in combination with bortezomib, melphalan and prednisone, wherein the pharmaceutical composition is clinically proven for subcutaneous administration.

In some embodiment, she method results in reduced occurrence or severity of infusion related reactions (IRR) in a subject when compared to an intravenous administration of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6.

In some embodiments, the pharmaceutical composition comprises about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 30,000 U of rHuPH20.

In some embodiments, the pharmaceutical composition comprises about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6 and about 2,000 U/mL of rHuPH20.

In some embodiments, the pharmaceutical composition comprises one or more excipients.

In some embodiments, the one or more excipients is histidine, methionine, sorbitol or polysorbate-20 (PS-20), or any combination thereof.

In some embodiments, the pharmaceutical composition comprises
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine, at a pH of about 5.5-5.6.

In some embodiments, the pharmaceutical composition comprises about 10 mM histidine.

In some embodiments, the pharmaceutical composition comprises about 300 mM sorbitol.

In some embodiments, the pharmaceutical composition comprises about 0.04% (w/v) PS-20.

In some embodiments, the pharmaceutical composition comprises about mg/mL methionine.

In some embodiments, the pharmaceutical composition comprises
about 1,800 mg of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 30,000 U of rHuPH20;
about 10 mM histidine;
about 300 mM sorbitol;
about 0.04% (w/v) PS-20; and
about 1 mg/mL methionine, at a pH of about 5.6.

In some embodiments, the pharmaceutical composition comprises
about 120 mg/mL of the antibody that specifically binds CD38 comprising the HCDR1 of SEQ ID NO: 1, the HCDR2 of SEQ ID NO: 2, the HCDR3 of SEQ ID NO: 3, the LCDR1 of SEQ ID NO: 4, the LCDR2 of SEQ ID NO: 5 and the LCDR3 of SEQ ID NO: 6;
about 2,000 U/mL of rHuPH20;
about 10 mM histidine;
about 300 mM sorbitol;
about 0.04% (w/v) PS-20; and
about 1 mg/mL methionine, at a pH of about 5.6.

In some embodiments, the antibody that specifically binds CD38 comprises a heavy chain variable region (VH) of SEQ ID NO: 7 and a light chain variable region (VL) of SEQ ID NO: 8.

In some embodiments, the antibody that specifically binds CD38 is an IgG1 isotype.

In some embodiments, the antibody that specifically binds CD38 comprises a heavy chain (HC) of SEQ ID NO: 9 and a light chain (LC) of SEQ ID NO: 10.

In some embodiments, the antibody that specifically binds CD38 is daratumumab.

In some embodiments, the antibody that specifically binds CD38 is a biosimilar of DARZALEX® brand of daratumumab.

In some embodiments, the pharmaceutical composition comprising the antibody that specifically binds CD38 and rHuPH20 is administered at a dose of about 1,800 mg of the antibody that specifically binds CD38 and about 30,000 U of rHuPH20 once a week, once in two weeks, once in three weeks or once in four weeks.

In some embodiments, bortezomib is administered at a dose of about 1.3 mg/m² twice a week.

In some embodiments, melphalan is administered at a dose of about 9 mg/m² twice a week.

In some embodiments, prednisone is administered at a dose of about 60 mg/m² twice a week.

In some embodiments, the pharmaceutical composition, bortezomib, melphalan and prednisone are administered for one or more 6-week cycles.

In some embodiments, the pharmaceutical composition is administered once a week in cycle 1, once every three weeks in cycles 2-9 and thereafter once every four weeks.

In some embodiments, bortezomib is administered at a dose of about 1.3 mg/m² twice a week on weeks 1, 2, 4 and 5 in cycle 1 and thereafter once a week on weeks 1, 2, 4 and 5 in cycles 2-9.

In some embodiments, melphalan is administered at a dose of about 9 mg/m2 twice a week in cycles 1-9.

In some embodiments, prednisone is administered about 60 mg/m² twice a week in cycles 1-9.

In some embodiments, bortezomib is administered subcutaneously or intravenously.

In some embodiments, melphalan is administered orally.

In some embodiments, prednisone is administered orally.

In some embodiments, melphalan is self-administered.

In some embodiments, is self-administered.

In some embodiments, multiple myeloma is relapsed, refractory, or both relapsed and refractory.

In some embodiments, myeloma is newly diagnosed multiple myeloma.

In some embodiments, the subject is eligible for high dose chemotherapy (HDC) and stem cell transplant (SCT).

In some embodiments, SCT is autologous SCT (ASCT), allogenic SCT or syngeneic SCT.

In some embodiments, SCT is ASCT.

In some embodiments, HDC is melphalan.

While having described the invention in general terms, the embodiments of the invention will be further disclosed in the following examples that should not be construed as limiting the scope of the claims.

### Example 1. Development of Co-Formulations of Daratumumab and Hyaluronidase

Various co-formulations were evaluated in order to establish the overall physico-chemical stability and delivery of daratumumab and rHuPH20 in the co-formulated product. The impact of the concentrations of the active constituent and/or the excipients in the formulations was evaluated in some of the stability and/or animal studies (shelf stability, shaking stability and in pig infusion studies). Table 2 provides a summary of the formulations that have been used in various studies.

TABLE 2

| For- | | | | Sorbitol/ | PS20 | Met | |
|---|---|---|---|---|---|---|---|
| mula- | Daratumumab | rHuPH20 | His | Sucrose | (% | (mg/ | |
| tion | (mg/mL) | (U/mL) | (mM) | (mM) | w/v) | mL) | pH |
| 1 | 100 | 500 | 10 | 300 | 0.04 | 2 | 5.5 |
| 2 | 120 | 2000 | 10 | 300 | 0.04 | 1 | 5.6 |
| 3 | 100 | 500 | 10 | 300 | 0.0 | 2 | 5.5 |
| 4 | 100 | 500 | 10 | 300 | 0.01 | 2 | 5.5 |
| 5 | 100 | 500 | 10 | 300 | 0.02 | 2 | 5.5 |
| 6 | 100 | 500 | 10 | 300 | 0.06 | 2 | 5.5 |
| 7 | 100 | 0 | 10 | 200/ | 0.04 | 0 | 5.5 |
| 8 | 100 | 0 | 10 | 100/ | 0.04 | 0 | 5.5 |
| 9 | 100 | 50 | 10 | 300 | 0.04 | 1 | 5.5 |
| 10 | 100 | 500 | 10 | 300 | 0.04 | 1 | 5.5 |
| 11 | 100 | 2000 | 10 | 300 | 0.04 | 1 | 5.5 |
| 12 | 100 | 5000 | 10 | 300 | 0.04 | 1 | 5.5 |

His: histidine
Met: methionine

The ranges of the excipients and the active constituents in the tested formulations are shown in Table 3.

TABLE 3

| Formulation component | Range | |
|---|---|---|
| rHuPH20 | 0-2000 | U/mL |
| Daratumumab | 100-120 | mg/mL |
| Histidine | 10 | mM |
| Sorbitol | 100-300 | mM |
| Sucrose | 0-200 | mM |
| Polysorbate-20 (PS20) | 0.0-0.04% | (w/v) |
| Methionine | 0-2 | mg/mL |

The generated formulations were tested in various assays for their characteristics, including evaluation of sub-visible particles, micro flow imaging (MFI), size exclusion chromatography (SEC), capillary iso-electric focusing (cIEF), SDS-PAGE (non-reducing and reducing), peptide mapping, extractable volume, turbidity, osmolality, and pH.

Sub-visible particles (Sub-vis): Number of sub-visible particles sizes of ≥10 m or ≥25 m is usually aggregates of protein molecules and can be assayed by the light obscuration HIAC method whereby the solution is passed through a small orifice and the blockage of light provides the information on the particle size passing through.

MFI: An orthogonal to the light obscuration method, micro flow imaging (MFI) takes snapshot images of particles flowing through and re-converts back to the number of particles present in a particular volume of liquid. This method provides information about the large aggregates of proteins present in the solution.

SEC: A size exclusion chromatographic separation method whereby a column is used to distribute the molecules within the solution flowing through according to their broad size range. Monomers, aggregates and fragments elute at different times from the column and hence their relative proportions in a sample can be quantified using a standard UV detector.

cIEF: Capillary iso-electric focusing distributes the molecules according to the charge on the molecule and is a good indicator of the overall chemical stability. For example deamidation may result in a change in the charge of the molecule and thus would be picked up by this method. The method provides an idea of the total acidic, basic and intact % of molecules present in the solution.

SDS (reducing and non-reducing conditions): SDS method provides information on the physical stability of the molecule. SDS provides a measure of the intact, aggregated and fragmented species present in the solution. Non-reducing SDS provides information on the respective intact, aggregated and fragmented constituents of the antibody while reducing SDS (after disulfide disruption) provides the same information for the heavy and light chains of the antibody.

Peptide mapping: Peptide mapping is an essential technique for studying the primary structure of proteins. For recombinant protein pharmaceuticals, peptide mapping is used for the initial proof of structure characterization. Peptide mapping also provides information on post translational modifications such as deamidation, oxidation etc Extractable volume: The method provides information on the amount/volume of liquid that can be withdrawn from the vial after the respective time point.

Turbidity: A light scattering based method to evaluate the physical stability of the solution. An increase in the size of the particles or aggregates results in an increase in the light scattering signal and is hence picked up as turbidity (opalescence) of the solution. Turbidity is measured in Nephelometric Turbidity Units (NTU).

Osmolality: Provides a measure of the total osmotic activity which is dependent on the total true activity of the molecules (activity coefficient multiplied by concentration). The solution must be close to the osmolality of the serum to be injectable.

pH: Provides an idea of the overall stability and is important that the pH remains constant throughout the shelf life.

rHuPH20 enzymatic activity: The determination of hyaluronidase activity is based on the formation of a precipitate when hyaluronic acid (HA) binds with acidified serum.

The activity is measured by incubating hyaluronidase with HA for 30 minutes in a 96-well plate format at 37° C. and then precipitating the undigested HA with the addition of acidified serum. The resulting turbidity is measured at 640 nm and the decrease in turbidity resulting from enzymatic cleavage of the HA substrate is a measure of the hyaluronidase activity.

Shelf stability of Formulation 1 (100 mg/mL Daratumumab, 10 mM Histidine, 300 mM Sorbitol, 0.04% PS-20, 2 mg/mL Methionine, 500 U/mL PrHuh20, pH 5.5) was evaluated using assays described. Samples were put on stability in 25R vials (filled at 16 mL volume) at different temperatures (5, 25 and 40° C.) and vials were pulled for analysis using various assays at different time points (0, 1, 2, 3, 4, 5 and/or 6 months). Data indicates that the co-formulated product is stable under the storage conditions both with respect to the Daratumumab as well as rHuPH20 as indicated by various assays. The profile as observed for particles, color, turbidity, sec etc was very similar to well behaved stable antibodies and the data is comparable to the stability data of some commercial mAb formulations.

Table 4 shows the number of particles in Formulation 1 over time as assessed using HIAC.

Table 5 shows the number of particles in Formulation 1 over time as assessed using MFI.

Table 6 shows the pH of Formulation 1 over time.

Table 7 shows the turbidity of Formulation 1 over time.

Table 8 shows the proportion of high-molecular weight aggregates and low molecular weight fragments in Formulation 1 over time.

Table 9 shows the acidic and basic species in Formulation 1 over time as assessed using cIEF.

Table 10 shows the percent (%) purity of Formulation 1 over time as assessed using reduced SDS-PAGE.

Table 11 shows the percent (%) purity of Formulation 1 over time as assessed using non-reduced SDS-PAGE.

Table 12 shows the percent (%) bioactivity of daratumumab and enzyme activity of rhPH20 in Formulation 1 over time.

TABLE 4

| | Average Cumulative Count/mL of | | | | | |
|---|---|---|---|---|---|---|
| Storage | particle sizes ≥10 μM or ≥25 μM | | | | | |
| Temperature | 0 months | | 3 months | | 6 months | |
| (° C.) | ≥10 μm | ≥25 μm | ≥10 μm | ≥25 μm | ≥10 μm | ≥25 μm |
| 5 | 7.17 | 6.33 | 6.5 | 4.8 | 42.50 | 30.83 |
| 25 | 7.17 | 6.33 | 5.5 | 1.8 | 110.50 | 81.83 |
| 40 | 7.17 | 6.33 | 64.5 | 44.8 | | |

TABLE 5

| | 0 months | 3 months | | | 6 months | |
|---|---|---|---|---|---|---|
| Temperature | 5 | 5 | 25 | 40 | 5 | 25 |
| (° C.) | | | | | | |
| Particles/mL | 539 | 1155 1 | 3668 | 3371 | 686 | 3581 |
| ≥2 − <10 μm | | | | | | |
| ECD | | | | | | |
| Particles/mL | 9.3 | 39 | 93 | 80 | 36 | 105 |
| ≥10 − <25 μm | | | | | | |
| ECD | | | | | | |
| Particles/mL | 1.4 | 2.6 | 13 | 9.3 | 7.4 | 7.2 |
| ≥25 − <70 μm | | | | | | |
| ECD | | | | | | |
| Particles/mL | 0.5 | 0.2 | 0.2 | 0.2 | 0.0 | 0.5 |
| ≥70 μm ECD | | | | | | |

TABLE 6

| Storage | | | | | |
|---|---|---|---|---|---|
| Temperature | pH over Storage Time (months) | | | | |
| (° C.) | 0 m | 1 m | 2 m | 3 m | 6 m |
| 5 | 5.7 | NA | 5.7 | 5.7 | 5.6 |
| 25 | 5.7 | 5.7 | 5.7 | 5.7 | 5.6 |
| 40 | 5.7 | 5.7 | 5.6 | 5.7 | NA |

m: month
NA: not analyzed

TABLE 7

| Storage | | | | | |
|---|---|---|---|---|---|
| Temperature | Average NTU over Storage Time (months) | | | | |
| (° C.) | 0 m | 1 m | 2 m | 3 m | 6 m |
| 5 | 3.5 | | 3.5 | 3.5 | 3.4 |
| 25 | 3.5 | 3.5 | 3.6 | 3.7 | 4.6 |
| 40 | 3.5 | 4 | 5.5 | 8.2 | NA |

m: month
NTU: Nephelometric Turbidity Units
NA: not analyzed

TABLE 8

| Storage | Storage | | | |
|---|---|---|---|---|
| Time | Temperature | Percentage (%) of species | | |
| (months) | (° C.) | HMW | Monomer | LMW |
| 0 | 5 | 0.74 | 99.25 | 0.01 |
| | 25 | 0.74 | 99.25 | 0.01 |
| | 40 | 0.74 | 99.25 | 0.01 |
| 3 | 5 | 0.84 | 99.16 | 0.02 |
| | 25 | 1.11 | 98.75 | 0.14 |
| | 40 | 1.87 | 94.50 | 3.64 |
| 6 | 5 | 0.89 | 99.10 | NA |
| | 25 | 1.30 | 98.38 | NA |
| | 40 | NA | NA | NA |

HMW: high molecular weight species
LMW: low molecular weight species
NA: not analyzed

TABLE 9

| Storage | Storage | | | |
|---|---|---|---|---|
| Time | Temperature | % Main | % Acidic | % Basic |
| (months) | (° C.) | peak | peaks | peaks |
| 0 | 5 | 69.2 | 27.8 | 3 |
| | 25 | 69.2 | 27.8 | 3 |
| | 40 | 69.2 | 27.8 | 3 |
| 3 | 5 | 68.2 | 28.9 | 2.9 |
| | 25 | 63 | 32.8 | 4.2 |
| | 40 | 31.3 | 60.2 | 8.5 |
| 6 | 5 | 68.1 | 28.7 | 3.2 |
| | 25 | 55.4 | 38 | 6.6 |
| | 40 | NA | NA | NA |

NA: not analyzed

TABLE 10

| Storage | | | | | |
|---|---|---|---|---|---|
| Temperature | % Purity over Storage Time (in months) | | | | |
| (° C.) | 0 m | 1 m | 2 m | 3 m | 6 m |
| 5 | 98.16 | NA | 97.96 | 98 | 98.02 |
| 25 | 98.16 | 97.87 | 97.66 | 97.57 | 96.75 |
| 40 | 98.16 | 96.4 | 95 | 92.88 | NA |

m: month
NA: not analyzed

TABLE 11

| Storage | | | | | |
|---|---|---|---|---|---|
| Temperature | % Purity over Storage Time (in months) | | | | |
| (° C.) | 0 | 1 | 2 | 3 | 6 |
| 5 | 97.56 | | 97.64 | 97.64 | 97.5 |
| 25 | 97.56 | 97.27 | 96.97 | 96.62 | 95.63 |
| 40 | 97.56 | 94.84 | 92.61 | 89.37 | NA |

m: month
NA: not analyzed

TABLE 12

| | | Storage Time and Temperature | | |
|---|---|---|---|---|
| | | | 6 months | 6 months |
| Molecule | Assay | 0 months | 5° C. | 25° C. |
| Daratumumab | ADCC* | 102 | 103 | 83 |
| | CDC* | 95 | 101 | 93 |
| PH20 | Enzymatic | 574 | 600 or | 609 |
| | activity** | | 584? | |

*Percent control
**U/mL

Agitation (shaking) stability of the Formulation 1 was also assessed using the above assays to characterize the formulations and study the impact of PS concentrations by varying just PS-20 concentrations in that formulation (Formulations 1, 3, 4, 5 and 6 in Table 2 where PS-20 was varied to 0, 0.01, 0.02, 0.04, 0.06%). The data indicated that the co-formulation was stable under the shaking conditions both with respect to the Daratumumab as well as the enzyme as indicated by various assays. The profile as observed for particles, color, turbidity, sec etc was very similar to well behaved stable antibodies for all concentrations of PS but 0% (0% PS20 formulation had particles and was not stable) and the data was comparable to the stability data of some commercial mAb formulations (data not shown).

Shelf stability of Formulation 2 (120 mg/mL daratumumab, 10 mM Histidine, 300 mM Sorbitol, 0.04% PS20, 1 mg/mL Methionine, 2000U/mL rhuPH20, pH 5.6) was evaluated using assays described. Samples were put on stability in 25R vials filled at 13.27 mL volume with overfill (1500 mg dose) at different temperatures and vials were pulled for analysis using various assays as below. The collected data indicated that the co-formulated product is stable under the storage conditions both with respect to the daratumumab as wellrHuPH20. The profile as observed for particles, color, turbidity, sec etc was very similar to well behaved stable antibodies and the data was comparable to the stability data of some commercial mAb formulations. rhuPH20 is very susceptible at higher temperatures and loses all activity very fast when stored at 40° C. Table 13 shows the characteristics of the formulation.

TABLE 13

| | Storage Time, Temperature and relative | | |
|---|---|---|---|
| | humidity (RH) | | |
| | | 1 month | 1 month |
| | 0 months | 40° C./ | 25° C./ |
| Characteristics and/or assay | 5° C. | 75% RH | 60% RH |
| Average Cumulative Count/mL | 58.89 | 375.68 | 4285.81 |
| of particle sizes 2-10 μm | | | |
| Average Cumulative Count/mL | 3.34 | 12.96 | 1.98 |
| of particle sizes 10-25 μm | | | |
| Average Cumulative Count/mL | 1.23 | .49 | 12 |
| of particle sizes ≥25 μm | | | |
| PS20 (% w/v) | 0.038 | 0.02 | 0.03 |
| pH | 5.6 | 5.6 | 5.6 |
| Turbidity (NTU) | 5 | 11 | 6 |
| % Purity (cSDS, reducing) | 98.4 | 98.6 | 98.1 |
| % AGHC, aglycosylated heavy | 0.4 | 0.5 | 0.4 |
| chain (cSDS, reducing) | | | |
| % Purity (cSDS, non-reducing) | 97.7 | 95.2 | 97.7 |
| % monomer, SE-HPLC | 99.1 | 98.0 | 98.8 |
| % aggregate, SE-HPLC | 0.9 | 1.6 | 1.1 |
| % fragments SE-HPLC | <0.10 | 0.4 | <0.10 |
| Daratumumab bioactivity, | 105 | 88 | 99 |
| CDC (% control) | | | |
| Daratumumab bioactivity, | 99 | 73 | 103 |
| ADCC (% control) | | | |
| rhuPH20 activity (U/mL) | 2205 | 0 | 2258 |

Formulations 3-8 were tested for their shelf stability or shaking stability using some or all assays described. The data indicated that the Formulations 3-8 were stable under the conditions assessed both with respect to the daratumumab as well as HuPH20 (formulations 7 and 8 had no rHuPH20). Methionine was included into formulations 1-6 and 9-12 to provide added oxidation stability. The profile as observed for particles, color, turbidity, sec etc was very similar to well behaved stable antibodies and the data was comparable to the stability data of some commercial mAb formulations (data not shown).

Agitation (shaking) stability of the Formulation 1 was also assessed using the above assays to characterize the formulations and study the impact of PS20 concentrations by varying just PS20 concentrations in that formulation (Formulations 1, 3, 4, 5 and 6 in Table 2 where PS20 was varied to 0, 0.01, 0.02, 0.04, 0.06%). The data indicated that the co-formulation was stable under the shaking conditions both with respect to daratumumab as well as rHuPH20 as indicated by various assays. The profile as observed for particles, color, turbidity, sec etc was very similar to well behaved stable antibodies for all concentrations of PS but 0% and the data was comparable to the stability data of some commercial mAb formulations (data not shown).

### Example 2. A Phase 3 Randomized, Multicenter Study of Subcutaneous Vs. Intravenous Administration of Daratumumab in Subjects with Relapsed or Refractory Multiple Myeloma

This is a Phase 3, randomized, open-label, active-controlled, multicenter study to demonstrate that the efficacy and pharmacokinetics of Dara-SC are not inferior to those for Dara-IV. The study population will consist of adults diagnosed with multiple myeloma who have received at least 3 prior lines of therapy including a PI and an IMiD, or whose disease is refractory to both a PI and an IMiD. Approximately 480 subjects will be assigned randomly to the Dara-SC group or the Dara-IV group in a 1:1 ratio. The randomization will be stratified by body weight at baseline (≤65 kg, 66 kg to 85 kg, >85 kg), number of prior lines of therapy (≤prior lines versus >4 prior lines), and type of myeloma (IgG versus non-IgG).

The study consists of 3 phases: a Screening Phase, a Treatment Phase, and a Follow-up Phase. The Screening Phase will be up to 28 days before randomization. The Treatment Phase will extend from randomization until discontinuation of study treatment. Each subject will be treated until the sponsor confirms that disease progression has occurred for that subject, the subject has unacceptable toxicity, or other reasons. The Follow-up Phase begins immediately following the End-of-Treatment Visit, and will continue until death, loss to follow up, withdrawal of consent for study participation, or end of study, whichever occurs first.

Treatment cycles are 28 days in length. The dosing schedule for both groups will be weekly for Cycles 1 and 2, every 2 weeks for Cycles 3 to 6, and every 4 weeks thereafter. Subjects who are assigned to the Dara-SC group will receive a fixed dose of Dara-SC 1800 mg (daratumumab 1800 mg co-formulated with rHuPH20 2000 U/mL).

Dara-SC will be delivered by SC injection in the abdominal SC tissue in left/right locations, alternating between individual doses. All subjects in the Dara-SC group will be observed for at least 6 hours after the end of the SC injection during Cycle 1 Day 1 and, if deemed necessary by the investigator, after consecutive injections. Subjects who are assigned to the Dara-IV group will receive Dara-IV 16 mg/kg by IV infusion pump.

### Primary Objectives

To show that SC administration of daratumumab co-formulated with recombinant human hyaluronidase PH20 (Dara-SC) is non-inferior to IV administration of daratumumab (Dara-IV) in terms of the overall response rate (ORR)
To show that Dara-SC is non-inferior to Dara-IV in terms of the maximum trough concentration (C_{trough})

### Secondary Objectives

To assess the pharmacokinetics and immunogenicity of Dara-SC and Dara-IV
To evaluate the safety of Dara-SC and Dara-IV
To evaluate the clinical benefit of Dara-SC and Dara-IV
To evaluate the immunogenicity of rHuPH20 following Dara-SC administration
To evaluate patient-reported satisfaction with Dara-SC and Dara-IV

### Primary Endpoints

The co-primary endpoints of this study are:
ORR, defined as the proportion of subjects with a PR or better according to the International Myeloma Working Group (IMWG) response criteria
Maximum C_{trough}, defined as the serum predose concentration of daratumumab on Cycle 3 Day 1
ORR: The number and proportion of subjects who achieve PR or better will be calculated for each group. The primary analysis will use the non-inferiority test for non-unity null according to Farrington and Manning Stat Med. 9:1447-1454, 1990. The relative risk and its two-sided 95% CI will be provided. If the lower bound of the 95% CI is ≥60%, the non-inferiority of Dara-SC relative to Dara-IV will be concluded. If non-inferiority in ORR is established and the lower limit of the 95% CI of the relative risk is ≥100%, the superiority of Dara-SC relative to Dara-IV will be concluded. The primary analysis will occur approximately 6 months after 480 subjects have been randomized

### Secondary Endpoints

Rate of IRRs
PFS, defined as the time from randomization to the date of disease progression or death due to any cause, whichever occurs first
Rate of VGPR or better, according to the IMWG response criteria
Rate of CR or better, according to the IMWG response criteria
Time to next therapy (TNT), defined as the time from randomization to the start of the first subsequent anti-cancer therapy
OS, defined as the time from randomization to the date of death
Patient-reported satisfaction with therapy, defined as the mean of responses to 7 of 9 questions in the modified Cancer Therapy Satisfaction Questionnaire (modified-CTSQ)
Duration of response, defined as date of onset of first response until date of disease progression or death
Time to response, defined as the time from randomization until onset of first response
IRR: The proportion of subjects who have an IRR and the 95% CI will be calculated for each treatment group. The IRR rate will be compared between the 2 groups using the stratified Cochran-Mantel-Haenszel test. The Mantel-Haenszel odds ratio will be provided along with its 2-sided 95% CI.
PFS: The median PFS and 95% CI in each treatment group will be estimated using the Kaplan-Meier method. The PFS distributions between the 2 treatment groups will be compared using the stratified log-rank test. The treatment effect (hazard ratio) and its two-sided 95% CI will be estimated using a stratified Cox regression model with treatment as the sole explanatory variable.
VGPR or better: The proportion of subjects who have a VGPR or better and the 95% CI will be calculated for each treatment group. The rate of VGPR or better will be compared between the 2 treatment groups using the stratified Cochran-Mantel-Haenszel test. The Mantel-Haenszel odds ratio will be provided along with its 2-sided 95% CI.
CR or better: The proportion of subjects who have a CR or better and the 95% CI will be calculated for each treatment group. The rate of CR or better will be compared between the 2 treatment groups using the stratified Cochran-Mantel-Haenszel test. The Mantel-Haenszel odds ratio will be provided along with its two-sided 95% CI.
TNT: The median TNT and 95% CI in each treatment group will be estimated using the Kaplan-Meier method. The TNT distributions will be compared between the 2 treatment groups using the stratified log-rank test. The treatment effect (hazard ratio) and its two-sided 95% CI will be estimated using a stratified Cox regression model with treatment as the sole explanatory variable.
OS: The median OS and 95% CI in each treatment group will be estimated using the Kaplan-Meier method. The OS distributions will be compared between the 2 treatment groups using the stratified log-rank test. The treatment effect (hazard ratio) and its 2-sided 95% CI will be estimated using a stratified Cox regression model with treatment as the sole explanatory variable.
Duration of response: A descriptive summary for duration of response will be provided. No statistical comparison will be made.
Time to response: A descriptive summary for time to response will be provided. No statistical comparison will be made.

Assessment of disease response will be conducted in accordance with the IMWG response criteria using a computerized algorithm. Efficacy assessments will include: monoclonal paraprotein (M-protein) measurements (serum and urine), serum free light chain (FLC), examination of bone marrow aspirate, skeletal survey, documentation of extramedullary plasmacytomas, and serum calcium corrected for albumin. Safety evaluations will include adverse event monitoring, physical examinations, electrocardiogram (ECG) monitoring, SC injection site evaluations, clinical laboratory parameters (hematology and chemistry), vital sign measurements, and Eastern Cooperative Oncology Group (ECOG) performance status. The National Cancer Institute-Common Terminology Criteria for Adverse Events (NCI-CTCAE) Version 4.03 will be used to grade toxicity throughout the study. Blood samples will be drawn for assessment of pharmacokinetic and biomarker parameters. If a fresh bone marrow aspirate is collected at Screening, a portion will be sent to a central laboratory for DNA/RNA sequencing. If feasible, a bone marrow aspirate will be collected from subjects at disease progression to evaluate mechanisms of daratumumab resistance.

### Statistical Methods

In a previous clinical study (MMY2002), of 106 subjects with relapsed or refractory multiple myeloma who had received at least 3 prior therapies and who were treated with Dara-IV 16 mg/kg, an ORR of 29.2% (95% CI: 20.8%, 38.9%) was observed. Non-inferiority of Dara-SC to Dara-IV in the current study is defined using a 60% retention of the lower bound (20.8%) of the 95% CI from Study MMY2002. With a planned 1:1 randomization, 480 subjects (n=240 in the Dara-SC group and n=240 in the Dara-IV group) will be needed to demonstrate non-inferiority with a power of 80% and a one-sided alpha=0.025, assuming that the true ORR is the same for both groups.

The study is also designed to establish non-inferiority of maximum C_{trough} between Dara-SC and Dara-IV. Dara-SC will be considered non-inferior to Dara-IV if the lower bound of the 90% confidence interval for the ratio of the geometric means of C_{trough} on Cycle 3 Day 1 is at least 80% (non-inferiority margin of 20%). A one-sided test is selected based on previous analyses that demonstrated a strong relationship between maximum C_{trough} and efficacy. However, there is no apparent relationship between drug exposure in the therapeutic dose range and adverse events of interest. With the planned 1:1 randomization, 480 subjects, and a one-sided alpha of 0.05, the power will be >95%. This assumes a true ratio of the maximum C_{trough} of 1 and a coefficient of variation of 0.6.

### Topline Results Summary

The following results are based on the primary analyses with a clinical cut-off date of 8 Jan. 2019.

A total of 522 subjects (Dara SC: 263; Dara IV: 259) were randomized. The median treatment duration was 6 cycles, and the median duration of follow-up was 7.46 months. The median duration of injection for subjects in Dara SC group was notably shorter than median duration of infusion for subjects in Dara IV group (5 mins for Dara SC; 421 mins, 255 mins, and 205 mins for the first, second and subsequent administrations of Dara IV, respectively).

Efficacy: ORR based on computerized algorithm was 41.1% (108/263) for Dara SC and 37.1% (96/259) for Dara IV. The estimate of the relative risk of Dara SC to Dara IV and 2-sided 95% CI were 1.11 (0.89, 1.37). This indicated that Dara SC retained at least 89% of the benefit of Dara IV with 97.5% confidence, meeting the non-inferiority criteria in efficacy. All pre-planned sensitivity analyses, including per-protocol analysis (relative risk=1.14 with 95% CI [0.92, 1.41]) and investigator assessed response, and subgroup analyses showed consistent results.

PK: The ratio of geometric means of maximum C_{trough} for Dara SC over Dara IV and 90% CI were 107.93% (95.74%-121.67%). The lower limit of the 90% CI (95.74%) was greater than 80%, meeting the PK non-inferiority criteria.

Since both endpoints were met, the non-inferiority of Dara SC to Dara IV was demonstrated in this study.

### Key Secondary Endpoints

Pre-specified hierarchical superiority testing was performed in the following sequential order: rate of IRRs, PFS, rate of VGPR or better, and OS. Rate of IRRs showed the superiority of Dara SC to Dara IV, while PFS and rate of VGPR or better showed similar results for Dara SC and Dara IV. OS data were not mature with a median duration of follow-up of 7.46 months.
Rate of IRRs: Dara SC treatment resulted in significantly lower rate of IRRs (Dara SC: 12.7%; Dara IV: 34.5%; odds ratio=0.28 with 95% CI: [0.18, 0.44]; p<0.0001).
PFS: Median PFS was similar for Dara SC and Dara IV groups (Dara SC: 5.59 months; Dara IV: 6.08 months; hazard ratio=0.99 with 95% CI: [0.78, 1.26]; pvalue=0.9258).
Rate of VGPR or better: Rate of VGPR or better was similar for Dara SC and Dara IV groups (Dara SC: 19.0%; Dara IV: 17.0%; odds ratio=1.16 with 95% CI: [0.73, 1.85]; p-value=0.5280).
OS: As of the data cutoff, OS data were not mature with a median duration of follow-up of 7.46 months. Similar number of deaths were observed in both treatment groups [Dara SC: 45 (17.1%); Dara IV: 48 (18.5%)]. The hazard ratio was 0.90 with 95% CI of (0.59, 1.35), and p-value=0.6032.

### Other Secondary Efficacy Endpoints:

TTR: Median time to first response was similar in both treatment groups (1.02 mons).
DOR: Median DOR were not reached in both Dara SC and Dara IV treatment groups.
The incidence rates of TEAEs were similar for Dara SC and Dara IV treatment groups Death within 30 days of last dose was similar (Dara SC: 5.0%; Dara IV: 7.0%). The incidence rate of TEAE leading to study drug discontinuation was also similar (Dara SC: 6.9%; Dara IV: 8.1%).
The incidence rate of IRRs was significantly lower in Dara SC (12.7%) than Dara IV (34.5%). IRRs were mainly reported as Grade 1 or Grade 2 and were mostly reported as associated with the first administration of study drug. 1.5% of subjects in Dara SC and 5.4% of subjects in Dara IV were reported Grade 3 IRRs, no Grade 4 IRRs were reported for both treatment groups.
The incidence rate of local injection-site reactions for subjects in Dara SC group was 6.9%, and all were reported as Grade 1 or Grade 2.

### Conclusion

Dara SC had demonstrated the non-inferiority to Dara IV with regards to the co-primary endpoints of ORR and maximum C_{trough}. The treatment groups showed similar results in key secondary efficacy endpoints including rate of VGPR or better, PFS and OS. Dara SC showed improved safety with significantly lower rate of IRR than Dara IV. Other safety profile was similar for both treatment groups. Overall, the study data demonstrated the comparable efficacy/safety profile of Dara SC in patients with relapsed or refractory multiple myeloma with the approved Dara IV product, with much reduced rate of IRR and improved convenience from substantially shorter infusion time.

### Results

The following results are based on the primary analyses with a clinical cut-off date of 8 Jan. 2019.

### Subject and Treatment Information

### Subject Disposition

518 of 522 randomized subjects were treated (Dara SC: 260; Dara IV: 258). At the time of the clinical cut-off, 42.7% of subjects in Dara SC and 43.0% in Dara IV subjects were still on study treatment. The main reasons for treatment discontinuation were progressive disease (Dara SC: 43.1%; Dara IV: 44.2%) and adverse event (Dara SC: 6.9%; Dara IV: 8.1%). 19.8% of the subjects in Dara SC group discontinued the study compared with 22.4% in Dara IV group. Table 14 shows the summary of subject treatment disposition, safety analysis population; Table 15 shows the summary of subject study disposition, intent-to-treat analysis population.

TABLE 14

| | Dara IV | Dara SC | Total |
|---|---|---|---|
| Analysis set: safety | 258 | 260 | 518 |
| Subjects who are still on | 111 (43.0%) | 111 (42.7%) | 222 (42.9%) |
| treatment | | | |
| Subjects who discontinued | 147 (57.0%) | 149 (57.3%) | 296 (57.1%) |
| treatment | | | |

Reason for discontinuation

| | | | |
|---|---|---|---|
| Adverse event | 21 (8.1%) | 18 (6.9%) | 39 (7.5%) |
| Death | 3 (1.2%) | 2 (0.8%) | 5 (1.0%) |
| Physician decision | 4 (1.6%) | 9 (3.5%) | 13 (2.5%) |
| Progressive disease | 114 (44.2%) | 112 (43.1%) | 226 (43.6%) |
| Withdrawal by subject | 5 (1.9%) | 7 (2.7%) | 12 (2.3%) |
| Other | 0 | 1 (0.4%) | 1 (0.2%) |

Note:
Dara IV = daratumumab intravenous;
Dara SC = daratumumab subcutaneous + recombinant human hyaluronidase PH20 (rHuPH20).
Note:
Percentages are calculated with the number of subjects in each treatment group as the denominators.

TABLE 15

| Dara IV | Dara SC | | Total |
|---|---|---|---|
| Analysis set: intent-to-treat | 259 | 263 | 522 |
| Subjects who discontinued | 58 (22.4%) | 52 (19.8%) | 110 (21.1%) |
| study | | | |
| Reason for discontinuation | | | |
| Death | 48 (18.5%) | 44 (16.7%) | 92 (17.6%) |
| Lost to follow-up | 1 (0.4%) | 1 (0.4%) | 2 (0.4%) |
| Withdrawal by subject | 8 (3.1%) | 4 (1.5%) | 12 (2.3%) |
| Other | 1 (0.4%) | 3 (1.1%) | 4 (0.8%) |

Note:
Dara IV = daratumumab intravenous;
Dara SC = daratumumab subcutaneous + recombinant human hyaluronidase PH20 (rHuPH20).
Note:
Percentages are calculated with the number of subjects in each treatment group as the denominators.

### Demographic and Baseline Disease Characteristics

Demographic and baseline disease characteristics were well balanced between the 2 treatment groups. The median age was 67.0 (range 33-92) years old, with 20.3% of the subjects ≥75 years of age. The median baseline body weight was 72.6 kg (range 28.6-138.0 kg). The majority of the subjects were white (78.2%) and had an ECOG performance score of 0 or 1 (83.5%).

The median number of lines of prior therapy was 4 lines. 33.8% of subjects were reported as ISS Stage I, 36.5% as Stage II, and 29.8% as Stage III. The majority of subjects had measurable disease in serum only (53.8%) with IgG (41.8%) and IgA (10.7%). 16.7% of the subjects had a high-risk cytogenetic abnormality.

The types of prior therapies for MM were similar for Dara SC and Dara IV treatment groups. All the subjects had taken prior systemic therapy, 50.8% of subjects had autologous stem cell transplant (ASCT). 100% of subjects were previously treated with both PI(s) and IMiD(s). Most subjects were refractory to a prior systemic therapy, including both PI and IMiD (49.4%), PI only (9.4%), IMiD only (28.4%), and neither PI or IMiD (12.8%). Table 16, Table 17 and Table 18 show the summary of patient demographics in the intent-to-treat group.

TABLE 16

| | Dara IV | Dara SC | Total |
|---|---|---|---|
| Analysis set: | 259 | 263 | 522 |
| intent-to-treat | | | |

Age (years)

| | | | |
|---|---|---|---|
| N | 259 | 263 | 522 |

Category, n (%)

| | | | |
|---|---|---|---|
| 18-<65 | 100 (38.6%) | 121 (46.0%) | 221 (42.3%) |
| 65-<75 | 100 (38.6%) | 95 (36.1%) | 195 (37.4%) |
| ≥75 | 59 (22.8%) | 47 (17.9%) | 106 (20.3%) |
| Mean (SD) | 66.8 (10.16) | 65.3 (9.11) | 66.1 (9.66) |
| Median | 68.0 | 65.0 | 67.0 |
| Range | (33; 92) | (42; 84) | (33; 92) |

Sex, n (%)

| | | | |
|---|---|---|---|
| N | 259 | 263 | 522 |
| Male | 149 (57.5%) | 136 (51.7%) | 285 (54.6%) |
| Female | 110 (42.5%) | 127 (48.3%) | 237 (45.4%) |

Race, n (%)

| | | | |
|---|---|---|---|
| N | 259 | 263 | 522 |
| White | 201 (77.6%) | 207 (78.7%) | 408 (78.2%) |
| Black or | 5 (1.9%) | 9 (3.4%) | 14 (2.7%) |
| African | | | |
| American | 40 (15.4%) | 32 (12.2%) | 72 (13.8%) |
| Asian | | | |
| American | 0 | 1 (0.4%) | 1 (0.2%) |
| Indian or | | | |
| Alaska Native | | | |
| Native | 1 (0.4%) | 0 | 1 (0.2%) |
| Hawaiian or | | | |
| other Pacific | | | |
| Islander | | | |
| Not Reported | 12 (4.6%) | 14 (5.3%) | 26 (5.0%) |

Ethnicity, n (%)

| | | | |
|---|---|---|---|
| N | 259 | 263 | 522 |
| Hispanic or | 9 (3.5%) | 14 (5.3%) | 23 (4.4%) |
| Latino | | | |
| Not Hispanic | 227 (87.6%) | 225 (85.6%) | 452 (86.6%) |
| or Latino | | | |
| Not Reported | 23 (8.9%) | 24 (9.1%) | 47 (9.0%) |
| Weight (kg) | | | |
| N | 258 | 262 | 520 |
| Category, | | | |
| n (%) | | | |
| ≤65 | 92 (35.7%) | 94 (35.9%) | 186 (35.8%) |
| >65-85 | 105 (40.7%) | 102 (38.9%) | 207 (39.8%) |
| >85 | 61 (23.6%) | 66 (25.2%) | 127 (24.4%) |
| Mean (SD) | 73.72 (17.864) | 74.55 (18.240) | 74.14 (18.042) |
| Median | 73.00 | 72.40 | 72.60 |
| Range | (28.6; 138.0) | (39.0; 130.0) | (28.6; 138.0) |
| Height (cm) | | | |
| N | 259 | 263 | 522 |
| Mean (SD) | 164.49 (11.173) | 164.27 (10.813) | 164.38 (10.983) |
| Median | 165.00 | 165.00 | 165.00 |
| Range | (125.4; 190.0) | (140.0; 194.0) | (125.4; 194.0) |
| Baseline | | | |
| ECOG score, | | | |
| n (%) | | | |
| N | 259 | 263 | 522 |
| 0 | 88 (34.0%) | 64 (24.3%) | 152 (29.1%) |
| 1 | 132 (51.0%) | 152 (57.8%) | 284 (54.4%) |
| 2 | 38 (14.7%) | 47 (17.9%) | 85 (16.3%) |
| >2ª | 1 (0.4%) | 0 | 1 (0.2%) |

Note:
Dara IV = daratumumab intravenous;
Dara SC = daratumumab subcutaneous + recombinant human hyaluronidase PH20 (rHuPH20).
^{a}1 subject who met the eligibility criteria with ECOG score of 1 at screening was assessed with ECOG performance score of 3 at Cycle 1 Day 1 as the baseline.
Note:
Corticosteroids are to be converted to dexamethasone with the following conversion factor:
1 mg hydrocortisone = 0.04 mg dexamethasone;
1 mg methylprednisolone = 0.19 mg dexamethasone;
1 mg prednisone = 0.15 mg dexamethasone;
1 mg prednisolone = 0.15 mg dexamethasone;
1 mg betamethasone = 1.25 mg dexamethasone;
1 mg deflazacort = 0.1 mg dexamethasone.

TABLE 17

| | Dara IV | Dara SC | Total |
|---|---|---|---|
| Analysis set: intent-to-treat | 259 | 263 | 522 |

Type of myeloma by immunofixation or serum FLC assay, n (%)

| | | | |
|---|---|---|---|
| N | 259 | 263 | 522 |
| IgG | 144 (55.6%) | 156 (59.3%) | 300 (57.5%) |
| IgA | 45 (17.4%) | 45 (17.1%) | 90 (17.2%) |
| IgM | 0 | 2 (0.8%) | 2 (0.4%) |
| IgD | 2 (0.8%) | 4 (1.5%) | 6 (1.1%) |
| IgE | 0 | 0 | 0 |
| Light chain | 62 (23.9%) | 53 (20.2%) | 115 (22.0%) |
| Kappa | 45 (17.4%) | 27 (10.3%) | 72 (13.8%) |
| Lambda | 15 (5.8%) | 23 (8.7%) | 38 (7.3%) |
| FLC-Kappaª | 1 (0.4%) | 2 (0.8%) | 3 (0.6%) |
| FLC-Lambda^{b} | 1 (0.4%) | 1 (0.4%) | 2 (0.4%) |
| Biclonal | 6 (2.3%) | 3 (1.1%) | 9 (1.7%) |

Type of measurable disease^{c}, n (%)

| | | | |
|---|---|---|---|
| N | 259 | 263 | 522 |
| Serum only | 137 (52.9%) | 144 (54.8%) | 281 (53.8%) |
| IgG | 109 (42.1%) | 109 (41.4%) | 218 (41.8%) |
| IgA | 25 (9.7%) | 31 (11.8%) | 56 (10.7%) |
| Other^{d} | 3 (1.2%) | 4 (1.5%) | 7 (1.3%) |
| Serum and urine | 45 (17.4%) | 47 (17.9%) | 92 (17.6%) |
| Urine only | 45 (17.4%) | 44 (16.7%) | 89 (17.0%) |
| Serum FLC only | 32 (12.4%) | 28 (10.6%) | 60 (11.5%) |

ISS Staging^{e}, n (%)

| | | | |
|---|---|---|---|
| N | 259 | 262 | 521 |
| I | 94 (36.3%) | 82 (31.3%) | 176 (33.8%) |
| II | 89 (34.4%) | 101 (38.5%) | 190 (36.5%) |
| III | 76 (29.3%) | 79 (30.2%) | 155 (29.8%) |

Note:
Dara IV = daratumumab intravenous;
Dara SC = daratumumab subcutaneous + recombinant human hyaluronidase PH20 (rHuPH20).
Note:
Percentages are calculated with the number of subjects in each group with available data as denominator.
^{a}Includes subjects without a positive immunofixation but with evidence of free light chain kappa by FLC testing.
^{b}Includes subjects without a positive immunofixation but with evidence of free light chain lambda by FLC testing.
^{c}Includes subjects without measurable disease in serum and urine.
^{d}Includes IgD, IgM, IgE and biclonal.
^{e}ISS staging is derived based on the combination of serum β2-microglobulin and albumin.

TABLE 18

| | | |
|---|---|---|
| Dara IV | Dara SC | Total |

Cytogenetic Risk^{f}

| | | | |
|---|---|---|---|
| N | 259 | 263 | 522 |
| Standard risk | 167 (64.5%) | 146 (55.5%) | 313 (60.0%) |
| High risk | 35 (13.5%) | 52 (19.8%) | 87 (16.7%) |
| Del(17p) | 22 (8.5%) | 32 (12.2%) | 54 (10.3%) |
| t(4; 14) | 15 (5.8%) | 22 (8.4%) | 37 (7.1%) |
| t(14; 16) | 4 (1.5%) | 7 (2.7%) | 11 (2.1%) |
| Not determined^{g} | 57 (22.0%) | 65 (24.7%) | 122 (23.4%) |

Number of lines of prior therapy, n (%)

| | | | |
|---|---|---|---|
| N | 259 | 263 | 522 |
| ≤4 Lines | 175 (67.6%) | 174 (66.2%) | 349 (66.9%) |
| >4 Lines | 84 (32.4%) | 89 (33.8%) | 173 (33.1%) |
| Mean (SD) | 4.3 (1.78) | 4.3 (1.72) | 4.3 (1.75) |
| Median | 4.0 | 4.0 | 4.0 |
| Range | (1; 15) | (2; 12) | (1; 15) |

Time since initial diagnosis to randomization (years)

| | | | |
|---|---|---|---|
| N | 259 | 263 | 522 |
| Mean (SD) | 6.14 (4.112) | 6.64 (3.823) | 6.39 (3.973) |
| Median | 5.36 | 6.01 | 5.57 |
| Range | (0.6; 39.0) | (0.8; 21.1) | (0.6; 39.0) |

Number of lytic bone lesions, n (%)

| | | | |
|---|---|---|---|
| N | 259 | 263 | 522 |
| None | 58 (22.4%) | 49 (18.6%) | 107 (20.5%) |
| 1-3 | 27 (10.4%) | 29 (11.0%) | 56 (10.7%) |
| 4-10 | 48 (18.5%) | 34 (12.9%) | 82 (15.7%) |
| More than 10 | 126 (48.6%) | 151 (57.4%) | 277 (53.1%) |

Presence of diffuse myeloma-related osteopenia, n (%)

| | | | |
|---|---|---|---|
| N | 259 | 263 | 522 |
| Yes | 118 (45.6%) | 126 (47.9%) | 244 (46.7%) |
| No | 141 (54.4%) | 137 (52.1%) | 278 (53.3%) |

Presence of extramedullary plasmacytomas, n (%)

| | | | |
|---|---|---|---|
| N | 259 | 263 | 522 |
| Yes | 18 (6.9%) | 17 (6.5%) | 35 (6.7%) |
| No | 241 (93.1%) | 246 (93.5%) | 487 (93.3%) |

Bone marrow % plasma cells, n (%)

| | | | |
|---|---|---|---|
| N | 255 | 255 | 510 |
| <10 | 64 (25.1%) | 53 (20.8%) | 117 (22.9%) |
| 10-30 | 112 (43.9%) | 107 (42.0%) | 219 (42.9%) |
| >30 | 79 (31.0%) | 95 (37.3%) | 174 (34.1%) |

Note:
Dara IV = daratumumab intravenous;
Dara SC = daratumumab subcutaneous + recombinant human hyaluronidase PH20 (rHuPH20).
Note:
Percentages are calculated with the number of subjects in each group with available data as denominator.
^{f}Cytogenetic risk is based on FISH or karyotyping.
^{g}Subjects who had either no cytogenetic testing performed or had cytogenetic testing performed but risk categorization could not be determined.

### Extent of Exposure

The median duration of treatment for subjects in Dara SC group (4.75 months) was similar to that in Dara IV group (5.36 months). The median relative dose intensity was high and similar for both treatment groups. The median duration of injection for subjects in Dara SC group was notably shorter than median duration of infusion for subjects in Dara IV group (5 mins for Dara SC; 421 mins, 255 mins, and 205 mins for the first, second and subsequent administrations of Dara IV, respectively). Treatment modification was less frequently reported in Dara SC group during dose administration compared with Dara IV group (0.8% vs. 36.0%), the most common reason for treatment modification in both treatment groups was due to adverse event.

### Co-Primary Endpoint Analysis

### Co-Primary Efficacy Endpoint Analysis on ORR

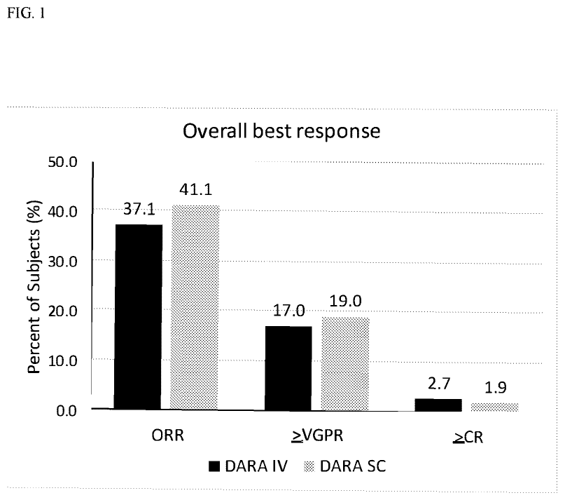

The Farrington-Manning (FM) estimate of the relative risk of Dara SC to Dara IV and 2-sided 95% CI were 1.11 (0.89, 1.37), obtained from FM test using 60% retention of ORR. The study met the clinical non-inferiority objective and demonstrated that Dara SC was non-inferior to Dara IV in efficacy in terms of ORR. Results based on per-protocol analysis set (relative risk=1.14 with 95% CI ([0.92, 1.41]), investigator assessed responses, and subgroup analysis showed consistent results. Table 19 and FIG. 1 shows the summary of the best overall response in the intent-to-treat population.

TABLE 19

| | Dara IV | | Dara SC | |
|---|---|---|---|---|
| | n (%) | 95% CI for %ª | n (%) | 95% CI for %ª |
| Analysis set: | 259 | | 263 | |
| intent-to-treat | | | | |

Best overall response

| | | | | |
|---|---|---|---|---|
| SCR | 2 (0.8%) | (0.1%, 2.8%) | 2 (0.8%) | (0.1%, 2.7%) |
| CR | 5 (1.9%) | (0.6%, 4.4%) | 3 (1.1%) | (0.2%, 3.3%) |
| VGPR | 37 (14.3%) | (10.3%, 19.1%) | 45 (17.1%) | (12.8%, 22.2%) |
| PR | 52 (20.1%) | (15.4%, 25.5%) | 58 (22.1%) | (17.2%, 27.6%) |
| MR | 28 (10.8%) | (7.3%, 15.2%) | 25 (9.5%) | (6.2%, 13.7%) |
| SD | 94 (36.3%) | (30.4%, 42.5%) | 102 (38.8%) | (32.9%, 45.0%) |
| PD | 27 (10.4%) | (7.0%, 14.8%) | 19 (7.2%) | (4.4%, 11.1%) |
| NE | 14 (5.4%) | (3.0%, 8.9%) | 9 (3.4%) | (1.6%, 6.4%) |
| Overall | 96 (37.1%) | (31.2%, 43.3%) | 108 (41.1%) | (35.1%, 47.3%) |
| response | | | | |
| (sCR + CR + | | | | |
| VGPR + PR) | | | | |
| CR or better | 7 (2.7%) | (1.1%, 5.5%) | 5 (1.9%) | (0.6%, 4.4%) |
| (SCR + CR) | | | | |
| VGPR or | 44 (17.0%) | (12.6%, 22.1%) | 50 (19.0%) | (14.5%, 24.3%) |
| better (SCR + | | | | |
| CR + VGPR) | | | | |

Note:
Dara IV = daratumumab intravenous;
Dara SC = daratumumab subcutaneous + recombinant human hyaluronidase PH20 (rHuPH20).
Key:
CI = confidence interval.
^{a}Clopper-Pearson exact confidence intervals are provided.
Note:
Percentages are calculated with the number of subjects in each group as denominators.
SCR: Stringent complete response
CR: Complete response
VGPR: Very good partial response
PR: Partial response
MR: Minimal response
SD: Stable disease
PD: Progressive disease
NE: Not evaluable
Overall response: SCR + CR + VGPR + PR
CR or better: SCR + CR
VGPR or better: SCR + CR + VGPR

### Co-Primary PK Endpoint Analysis on Maximum C_{trough}

The ratio of geometric means of maximum C_{trough} for Dara SC over Dara IV and 90% CI were 107.93% (95.74%-121.67%). The lower limit of the 90% CI (95.74%) was greater than 80%, therefore, it demonstrated that Dara SC was non-inferior to Dara IV in terms of maximum C_{trough} and the study met the PK non-inferiority objective.

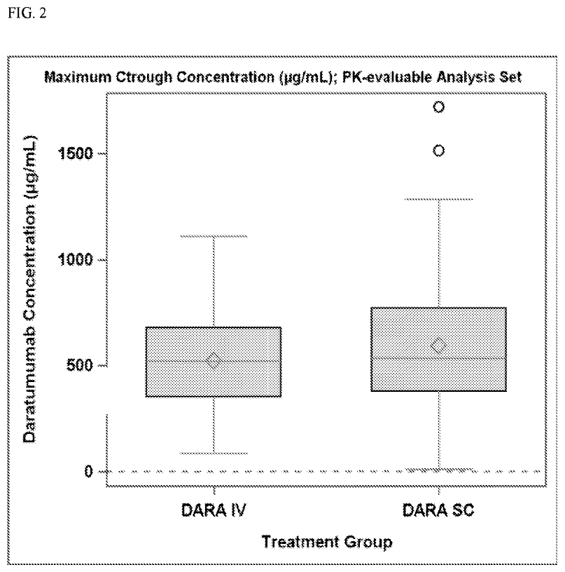

Both co-primary endpoints of ORR and maximum C_{trough} had met their non-inferiority criteria, therefore, the non-inferiority of Dara SC relative to Dara IV was demonstrated in the study. Table 20 and FIG. 2 shows the summary of maximum C_{trough} concentration (μg/mL) in pharmacokinetic-evaluable subjects.

TABLE 20

| | Dara IV | Dara SC |
|---|---|---|
| Analysis set: Pharmacokinetic-evaluable | 146 | 149 |

Maximum Ctrough Concentration
(μg/mL, pre-dose on Cycle 3 Day 1)

| | | |
|---|---|---|
| N T | 146 | 149 |
| Mean (SD) | 522 (226) | 593 (306) |
| Geometric meanª | 463 | 499 |
| Ratio (Dara SC/Dara IV) | | 107.93% |
| 90% CI | | (95.74%-121.67%) |
| Coefficient of variation | 43.2% | 51.6% |
| Median | 519 | 532 |
| Range | (86.1; 1109) | (9.97; 1720) |

Note:
Dara IV = daratumumab intravenous;
Dara SC = daratumumab subcutaneous + recombinant human hyaluronidase PH20 (rHuPH20).
^{a}Maximum C trough concentration data were natural log (ln) transformed prior to the calculation of geometric mean, the ratio of geometric mean and its 90% confidence interval, and then transformed back to the linear scale.
Note:
The ratio of geometric mean was calculated using Dara IV as the reference group.

### Major Secondary Endpoint(s) Analysis

Pre-specified hierarchical superiority testing was performed in the following sequential order: rate of IRRs, PFS, rate of VGPR or better, and OS. Table 19 shows the summary of response rate of VGPR or better in intent-to-treat analysis set.

### Rate of IRRs

Dara SC showed significantly lower rate and superiority on IRRs (12.7% vs. 34.5%), the stratified CMH estimate of odds ratio was 0.28 with 95% CI (0.18, 0.44) and p-value<0.0001.

### PFS

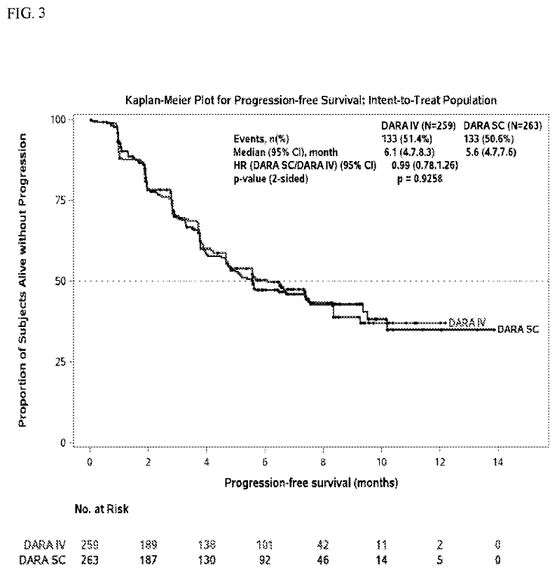

Median PFS was similar for Dara SC and Dara IV groups (Dara SC: 5.59 months; Dara IV: 6.08 months; hazard ratio=0.99 with 95% CI: [0.78, 1.26]; p-value=0.9258). FIG. 3 shows the Kaplan-Meier plot for PFS in intent-to-treat population.

### Rate of VGPR or Better

Rate of VGPR or better was similar for both treatment groups (Dara SC: 19.0%; Dara IV: 17.0%; odds ratio=1.16 with 95% CI: [0.73, 1.85]; p-value=0.5280).

### Overall Survival

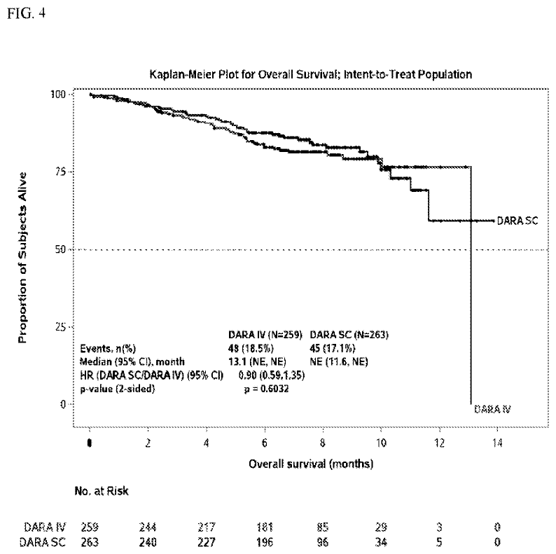

As of the data cutoff, OS data were not mature with a median duration of follow-up of 7.46 months. Similar number of deaths were observed for Dara SC and Dara IV treatment groups [Dara SC: 45 (17.1%); Dara IV: 48 (18.5%)]. The hazard ratio (Dara SC vs. Dara IV) was 0.90 with 95% CI of (0.59, 1.35), p-value=0.6032. FIG. 4 shows the Kaplan-Meier plot for overall survival (OS) in DARZALEX® (daratumumab) intravenous (DARA IV) and daratumumab subcutaneous (DARA SC) groups, intent-to-treat population

### Duration of Response

As of the data cutoff, similar number of responders who developed disease progression or died due to disease progression were observed for both treatment groups [Dara SC: 25 (23.1%); Dara IV: 20 (20.8%)], the median DOR were not reached in both Dara SC and Dara IV treatment groups.

### Time to Response

Median time to first response of PR or better was similar for both treatment groups (Dara SC: 1.02 months; Dara IV: 1.02 months).

### Safety

Dara SC and Dara IV exhibited similar safety profiles, except for the significantly lower incidence of IRRs in Dara SC. IRRs were generally reported as lower toxicity grade and most frequently reported as associated with the first administration of study drug.

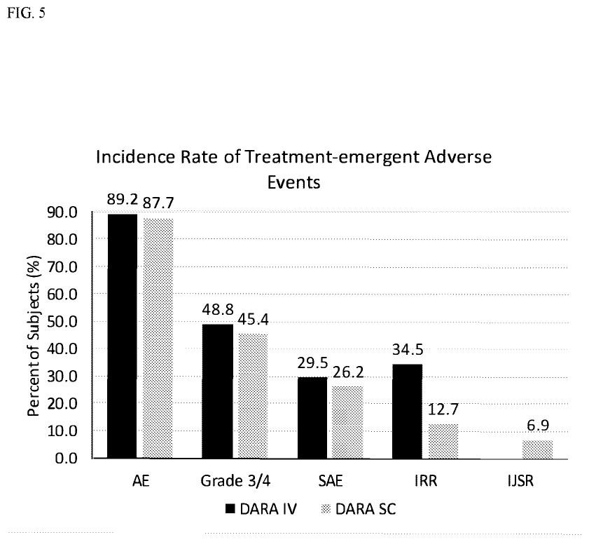

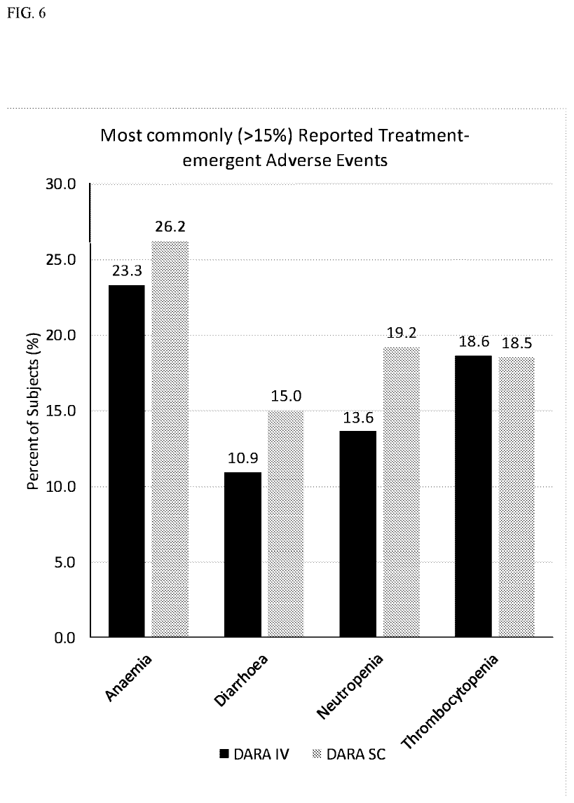

The incidence of local injection-site reactions for subjects in Dara SC group was 18 (6.9%) and all were reported as Grade 1 or Grade 2. Table 21 shows the summary of rate of treatment-emergent infusion-related reactions in the safety analysis population. Table 22 shows the summary of TE IRR by Grade 3 or 4 in the safety analysis population. FIG. 5 shows the graph of incidence rate of treatment-emergent adverse events in DARA IV and DARA SC groups. FIG. 6 shows the most commonly reported treatment-emergent adverse events in DARA IV and DARA SC groups.

TABLE 21

| | | | | | Odds | |
|---|---|---|---|---|---|---|
| | DARA IV | | DARA SC | | Ratio^{b} | |
| | | 95% CI | | 95% CI | (95% | |
| | n (%) | for %ª | n (%) | for %ª | CI) | P-value^{c} |
| Analysis set: | 258 | | 260 | | | |
| safety | | | | | | |
| Subjects | 89 | (28.7%, | 33 | (8.9%, | 0.28 | <0.0001 |
| who had | (34.5%) | 40.6%) | (12.7%) | 17.4%) | (0.18, | |
| treatment- | | | | | 0.44) | |
| emergent | | | | | | |
| infusion- | | | | | | |
| related | | | | | | |
| reactions | | | | | | |

Note:
Dara IV = daratumumab intravenous;
Dara SC = daratumumab subcutaneous + recombinant human hyaluronidase PH20 (rHuPH20).
Key: CI = confidence interval.
^{a}Clopper-Pearson exact confidence intervals are provided.
^{b}Stratified Cochran-Mantel-Haenszel estimate of the common odds ratio of Dara SC
over Dara IV is used. The stratification factors include body weight at baseline (≤65 kg, 66 kg to 85 kg, >85 kg), number of prior lines of therapy (≤4 prior lines versus >4 prior lines), and type of myeloma (IgG versus non-IgG).
^{c}P-value is from Cochran-Mantel-Haenszel Chi-Squared test.
Note:
Percentages are calculated with the number of subjects in each group as denominators.

TABLE 22
TSFAE04: Summary of Treatment-emergent Infusion-related Reactions
by Grade 3 or 4; Safety Analysis Set (Study 54767414MMY3012)

| | Dara IV | | | Dara SC | | |
|---|---|---|---|---|---|---|
| | N = 258 | | | N = 260 | | |
| Analysis set: safety | Any Grade | Grade 3 | Grade 4 | Any Grade | Grade 3 | Grade 4 |
| Total number of subjects | 89 (34.5%) | 14 (5.4%) | 0 | 33 (12.7%) | 4 (1.5%) | 0 |
| with treatment-emergent | | | | | | |
| infusion-related | | | | | | |
| reactions | | | | | | |

Note:
Percentages are calculated with the number of subjects in each group as denominators.

sequence listing

| | | SEQ ID |
|---|---|---|
| | Amino Acid Sequence | NO: |
| HCDR1 | SFAMS | 1 |
| HCDR2 | AISGSGGGTYYADSVKG | 2 |
| HCDR3 | DKILWFGEPVFDY | 3 |
| LCDR1 | RASQSVSSYLA | 4 |
| LCDR2 | DASNRAT | 5 |
| LCDR3 | QQRSNWPPTF | 6 |
| VH | EVQLLESGGGLVQPGGSLRLSCAVSGFTFNSFAMSWVRQAPGK | 7 |
| | GLEWVSAISGSGGGTYYADSVKGRFTISRDNSKNTLYLQMNSL | |
| | RAEDTAVYFCAKDKILWFGEPVFDYWGQGTLVTVSS | |
| VL | EIVLTQSPATLSLSPGERATLSCRASQSVSSYLAWYQQKPGQAP | 8 |
| | RLLIYDASNRATGIPARFSGSGSGTDFTLTISSLEPEDFAVYYCQQ | |
| | RSNWPPTFGQGTKVEIK | |
| HC | EVQLLESGGGLVQPGGSLRLSCAVSGFTFNSFAMSWVRQAPGK | 9 |
| | GLEWVSAISGSGGGTYYADSVKGRFTISRDNSKNTLYLQMNSL | |
| | RAEDTAVYFCAKDKILWFGEPVFDYWGQGTLVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVDK | |
| | RVEPKSCDKTHTCPPCPAPELLGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |
| LC | EIVLTQSPATLSLSPGERATLSCRASQSVSSYLAWYQQKPGQAP | 10 |
| | RLLIYDASNRATGIPARFSGSGSGTDFTLTISSLEPEDFAVYYCQQ | |
| | RSNWPPTFGQGTKVEIKRTVAAPSVFIFPPSDEQLKSGTASVVCL | |
| | LNNFYPREAKVQWKVDNALQSGNSQESVTEQDSKDSTYSLSST | |
| | LTLSKADYEKHKVYACEVTHQGLSSPVTKSFNRGEC | |
| IgG1 | ASTKGPSVFPLAPSSKSTSGGTAALGCLVKDYFPEPVTVSWNSG | 11 |
| constant | ALTSGVHTFPAVLQSSGLYSLSSVVTVPSSSLGTQTYICNVNHKP | |
| domain | SNTKVDKRVEPKSCDKTHTCPPCPAPELLGGPSVFLFPPKPKDTL | |
| | MISRTPEVTCVVVDVSHEDPEVKFNWYVDGVEVHNAKTKPREE | |
| | QYNSTYRVVSVLTVLHQDWLNGKEYKCKVSNKALPAPIEKTIS | |
| | KAKGQPREPQVYTLPPSREEMTKNQVSLTCLVKGFYPSDIAVE | |
| | WESNGQPENNYKTTPPVLDSDGSFFLYSKLTVDKSRWQQGNVF | |
| | SCSVMHEALHNHYTQKSLSLSPGK | |
| rHuPH20 | MGVLKFKHIFFRSFVKSSGVSQIVFTFLLIPCCLTLNFRAPPVIPN | 12 |
| | VPFLWAWNAPSEFCLGKFDEPLDMSLFSFIGSPRINATGQGVTIF | |
| | YVDRLGYYPYIDSITGVTVNGGIPQKISLQDHLDKAKKDITFYMP | |
| | VDNLGMAVIDWEEWRPTWARNWKPKDVYKNRSIELVQQQNV | |
| | QLSLTEATEKAKQEFEKAGKDFLVETIKLGKLLRPNHLWGYYLF | |
| | PDCYNHHYKKPGYNGSCFNVEIKRNDDLSWLWNESTALYPSIY | |
| | LNTQQSPVAATLYVRNRVREAIRVSKIPDAKSPLPVFAYTRIVFT | |
| | DQVLKFLSQDELVYTFGETVALGASGIVIWGTLSIMRSMKSCLL | |
| | LDNYMETILNPYIINVTLAAKMCSQVLCQEQGVCIRKNWNSSDY | |
| | LHLNPDNFAIQLEKGGKFTVRGKPTLEDLEQFSEKFYCSCYSTLS | |
| | CKEKADVKDTDAVDVCIADGVCIDAFLKPPMETEEPQIFYNASP | |
| | STLSATMFIVSILFLIISSVASL | |

We claim:

1. A method of achieving a non-inferior median progression-free survival (PFS) in a subject or a population of subjects with multiple myeloma, said method comprising subcutaneously administering to the subject or the population of subjects a pharmaceutical composition comprising:
an antibody that specifically binds CD38 and comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and
recombinant human hyaluronidase (rHuPH20),
wherein the non-inferior median PFS is relative to median PFS of a reference population of subjects with multiple myeloma, said reference population having been administered said pharmaceutical composition intravenously.

2. The method of claim 1, wherein the non-inferior median PFS is about 5.59 months.

3. The method of claim 1, wherein said method is of achieving a non-inferior PFS in a subject with multiple myeloma.

4. The method of claim 1, wherein said method is of achieving a non-inferior PFS in a population of subjects with multiple myeloma.

5. The method of claim 1, wherein the pharmaceutical composition is clinically proven for subcutaneous administration.

6. The method of claim 1, wherein the pharmaceutical composition comprises about 1,800 mg of said antibody and about 30,000 U rHuPH20.

7. The method of claim 6, wherein the pharmaceutical composition comprises about 120 mg/mL of said antibody and about 2,000 U/mL rHuPH20.

8. The method of claim 1, wherein the pharmaceutical composition comprises one or more excipients.

9. The method of claim 8, wherein at least one of said excipients is histidine, methionine, sorbitol or polysorbate-20 (PS-20), or any combination thereof.

10. The method of claim 9, wherein the pharmaceutical composition is at a pH of about 5.5-5.6, and wherein the pharmaceutical composition comprises:
between about 5 mM and about 15 mM histidine;
between about 100 mM and about 300 mM sorbitol;
between about 0.01% w/v and about 0.04% w/v PS-20; and
between about 1 mg/mL and about 2 mg/mL methionine.

11. The method of claim 10, wherein the pharmaceutical composition comprises about 10 mM histidine.

12. The method of claim 10, wherein the pharmaceutical composition comprises about 300 mM sorbitol.

13. The method of claim 12, wherein the pharmaceutical composition comprises about 0.04% (w/v) PS-20.

14. The method of claim 13, wherein the pharmaceutical composition comprises about mg/mL methionine.

15. The method of claim 14, wherein the pharmaceutical composition is at a pH of about 5.6, and wherein the pharmaceutical composition comprises:
about 1,800 mg of said antibody;
about 30,000 U of rHuPH20;
about 10 mM histidine;
about 300 mM sorbitol;
about 0.04% (w/v) PS-20; and
about 1 mg/mL methionine.

16. The method of claim 15, wherein the pharmaceutical composition is at a pH of about 5.6, and wherein the pharmaceutical composition comprises:
about 120 mg/mL of said antibody;
about 2,000 U/mL of rHuPH20;
about 10 mM histidine;
about 300 mM sorbitol;
about 0.04% (w/v) PS-20; and
about 1 mg/mL methionine.

17. The method of claim 1, wherein the antibody that specifically binds CD38 comprises a heavy chain variable region (VH) of SEQ ID NO: 7 and a light chain variable region (VL) of SEQ ID NO: 8.

18. The method of claim 1, wherein the antibody that specifically binds CD38 is an IgG1 isotype.

19. The method of claim 1, wherein the antibody that specifically binds CD38 comprises a heavy chain (HC) of SEQ ID NO: 9 and a light chain (LC) of SEQ ID NO: 10.

20. The method of claim 1, wherein the antibody that specifically binds CD38 is daratumumab.

21. The method of claim 1, wherein the antibody that specifically binds CD38 is a biosimilar of daratumumab.

22. The method of claim 1, wherein the pharmaceutical composition comprising the antibody that specifically binds CD38 and rHuPH20 is administered at a dose of about 1,800 mg once a week, about 1,800 mg once in two weeks, about 1,800 mg once in three weeks or about 1,800 mg once in four weeks.

23. The method of claim 22, wherein the pharmaceutical composition is administered for one or more 28-day cycles.

24. The method of claim 23, wherein the pharmaceutical composition is administered once a week in the first and the second 28-day cycle, once in two weeks in the third and the fourth 28-day cycle, and thereafter once in four weeks in any subsequent 28-day cycle.

25. The method of claim 1, wherein multiple myeloma is relapsed, refractory, or both relapsed and refractory multiple myeloma.

26. The method of claim 25, wherein the subject received at least three prior lines of therapy including a proteasome inhibitor (PI) and an immunomodulatory agent (IMiD) or who are refractory to both a PI and an IMiD.
